(19) United States
(12) Patent Application Publication
(10) Pub. No.: US 2010/0178279 A1
(43) Pub. Date: Jul. 15, 2010

(54) Beta-Glucan Enhances Hematopoietic Progenitor Cells Engraftment and Promotes Recovery from Chemotoxicity

(75) Inventors: Susanna Cunningham-Rundles, New York, NY (US); Hong Lin Smith-Jones, New York, NY (US)

Correspondence Address: SCULLY SCOTT MURPHY & PRESSER, PC, 400 GARDEN CITY PLAZA, SUITE 300, GARDEN CITY, NY, 11530, US

(73) Assignee: Cornell University, Ithaca, NY (US)

(21) Appl. No.: 12/688,160

(22) Filed: Jan. 15, 2010

Related U.S. Application Data
(60) Provisional application No. 61/144,920, filed on Jan. 15, 2009.
(60) Provisional application No. 61/239,609, filed on Sep. 3, 2009.

(51) Int. Cl. A61K 35/14; C12N 5/078; A61P 43/00

(52) U.S. Cl. 424/93.7; 435/372

(57) ABSTRACT

Methods to use beta glucans in the culture of cell populations containing CD34+ cells in order to expand the numbers of CD34+ subsets of progenitor and stem cells are provided. Methods to improve homing and engraftment of stem and progenitor cells by first culturing the cells with beta glucans, or co-administering with beta glucans, are also provided. Additionally, methods to ameliorate chemotherapy toxicity and promote development of functionally active neutrophils by administering beta glucans are presented. The beta glucans are preferably extracted from maitake mushroom (Grifola frondosa).

### CROSS-REFERENCE TO RELATED APPLICATIONS

This application claims the benefit of priority to U.S. Provisional Application No. 61/144,920, filed Jan. 15, 2009, and to U.S. Provisional Application No. 61/239,609 filed Sep. 3. 2009.

### STATEMENT REGARDING FEDERALLY SPONSORED RESEARCH OR DEVELOPMENT

This invention was made with United States Government support under Grant Numbers NIH NCI 29502: CNRU Pilot Study: (PI H. Lin) Effect of MBG (MBG) on hematopoietic expansion & engraftment of cord blood cells in NOD/SCID mouse; NIH NCI R25 105012 Collaborative Program in Nutrition and Cancer Prevention; H. Lin, Training and Career Development Fellowship Award; NIH NCCAM and ODS: 1-P50-AT02779 Botanical Research Center for Botanical Immunomodulators. The United States Government has certain rights in the invention.

### FIELD OF THE INVENTION

This invention generally relates to the use of beta glucans in expanding hematopoietic progenitor cells in culture, and in promoting homing and engrafting of hematopoietic progenitor cells to the bone marrow of a transplantation recipient. Additionally, the invention relates to the use of beta glucans to ameliorate toxicity of chemotherapy and to promote the development of neutrophils and their functional activity.

### BACKGROUND OF THE INVENTION

Beta glucans are cell wall constituents of yeast, fungi and bacteria as well as edible mushrooms and barley. Beta glucans are not expressed on mammalian cells and are recognized as pathogen-associated molecular patterns (PAMPS) by pattern recognition receptors (PRR), primarily the C-type lectin receptor dectin-1 and also interact via the complement receptor 3 (CR3) (1-4). Dectin 1 is a small type II transmembrane receptor with a lectin-like carbohydrate recognition domain, which recognizes beta1, 3- and beta1, 6-linked glucans and intact yeast, while CR3 is a widely expressed beta 2-integrin containing a lectin domain, which mediates carbohydrate recognition (1, 5). Both CR3 and dectin-1 expression are affected by bone marrow injury and have a role in the restorative effects of soluble beta glucan on hematopoiesis after radiation or chemotherapy (6, 7).

Studies in the mouse have shown that specific beta glucans such as PGG-glucan derived from yeast (Saccharomyces cerevisiae) or from mushrooms such as Grifola frondosa, Sclerotinia sclerotiorum and Sparassis crispa, can enhance hematopoiesis and protect bone marrow cells from radiation and chemotherapeutic injury (8-12). PGG-glucan (poly-1-6 beta-D-glucopyranosyl 1,3-beta-glucopyranose) has been shown to synergize with colony-stimulating growth factors leading to increased colony forming activity and to have direct effects on committed hematopoietic progenitor cells (10, 13, 14). Increase in colony growth factor production after intraperitoneal injection of SSG, a 1,3-beta-D-glucan obtained from the culture filtrate of S. sclerotiorum led to increases in both splenic hematopoiesis and peripheral leukocyte numbers (9). Administration of SCG, a 1,3-beta-D-glucan from Sparassis crispa to mice after cyclophosphamide treatment restored hematopoiesis and the effect was mediated by beta glucan binding to dectin-1 (7, 9, 15).

Human umbilical cord blood contains a rich population of primitive hematopoietic cells including lineage-restricted committed progenitors (HPC), and primitive uncommitted hematopoietic stem cells (HSC) that sustain multilineage hematopoiesis (17). HSC develop into all of the blood forming cells of the hematopoietic system while the myeloid restricted HPC are critical for the initial phase of clinical transplantation (18, 19). Functional assays are required to assess the biological activity of progenitor and stem cells (21). Committed myeloid progenitors (HPC) form discrete colonies of mature cells in response to hematopoietic cytokines in semi-solid medium and these cells are measured as colony-forming units (CFU) in validated CFU assays (22). Human CD34+ cells with HSC function are identified by in vivo functional assay in the NOD/SCID mouse by xenotransplantation assay (23). After brief exposure to irradiation the NOD/SCID mouse models can be repopulated with human cells over days to weeks and offer a validated approach to assess HSC homing and engraftment (23, 24). The severe combined immune deficient mouse repopulating cell (SRC) assay which measures relative SRC activity in the NOD/SCID mouse provides a clinically useful correlate for graft function (22). CXCR4, the G-protein coupled receptor that binds to stromal cell-derived factor-1 alpha (SDF-1) is an important determinant for CD34+ human precursor cell migration leading to homing and engraftment in the nonobese diabetic/severe combined immunodeficient (NOD/SCID) mouse assay for transplantation (25-27). CXCR4 expression on the surface of CD34+ precursor cells denotes very early-uncommitted HSC proliferation and homing and correlates with long-term culture-initiating activity (28).

Cord blood is emerging as an important source of progenitor cells for hematopoietic reconstitution in the treatment of both malignant and non-malignant blood diseases. Compared to bone marrow, cord blood stem cells cause less graft-versus-host disease (29, 30). However, cord blood is limited in precursor cell number, especially with smaller volume cord blood samples.

The demonstration that accelerated dose-dense chemotherapy with sequential doxorubicin/cyclophosphamide followed by paclitaxel significantly improved clinical outcomes in breast cancer has established a new standard of care and proven the Norton-Simon hypothesis that increased frequency of cytotoxic therapy is superior to dose escalation (71, 72). With sequential dosing, the requirement for growth factor support may be considered separately for each phase. Prophylactic granulocyte colony stimulating factor (G-CSF) is recommended with accelerated dose dense chemotherapy (73) but causes bone pain and can reduce the concentration of bone marrow progenitor cells over a significant period of time (74, 75). Suspending the use of G-CSF growth factor support during paclitaxel treatment after the doxorubicin and cyclophosphamide components of chemotherapy has been attempted (76). A recent study in the setting of accelerated paclitaxel treatment of early stage breast cancer showed that not giving prophylactic G-CSF was acceptable. While 40% of patients became neutropenic and 10% required secondary G-CSF, there were no treatment delays (76). However in another study of early breast cancer treatment in which G-CSF was held during the paclitaxel phase of chemotherapy, 40% of patients did not complete therapy on time due to dose delays (77). The patients who became neutropenic tended to be younger with a lower body surface area, to have lower absolute white blood cell (WBC) and lower absolute neutrophil counts (ANC) (77).

Paclitaxel is widely used in cancer including as first-line treatment of metastatic breast cancer (78-83). Paclitaxel was discovered in the National Cancer Institute (NCI) screening program as a natural product extract from the Pacific yew tree, Taxus brevifolia, with activity against a broad range of tumor types (100), especially breast, ovarian, and lung cancer. Ptx lacks cumulative toxicity (101) and is widely used both for anti-tumor activity and mobilization of peripheral blood stem cells in cancer patients (102). Although the mechanism of anti-cancer effect involves induction of tubulin polymerization preventing formation of the mitotic spindle, Ptx also causes apoptosis at doses that do not affect tubulin (103, 104). The primary toxicity of Ptx is leukopenia, mainly neutropenia (105). Pharmacodynamic studies in the rat have shown that time course of paclitaxel exposure affected critical parameters of hematopoiesis specifically the production, maturation, and lifespan of precursor cells and mature neutrophils (106). Related modeling studies in patients suggest that neutrophil progenitor cells remain sensitive to paclitaxel in the early maturating phase (107). Ptx reduces mesenchymal stem cell proliferation and causes a partial arrest of these cells at the G(2) phase of the cell cycle (108). The overall effect of Ptx treatment in the non tumor bearing host is acute hematotoxic injury that leads to stimulation of G-CSF, the major regulator of neutrophilic granulocytes (109) and to rebound leukocytosis. G-CSF and GM-CSF stimulate colony formation by primitive hematopoietic stem cells and can synergize with other growth factors such as IL-1 alpha to enhance recovery from chemotoxicity (110).

Few previous studies have examined the hematotoxic effects of paclitaxel (Ptx) in vivo in experimental models and none have assessed the dynamics of leukocyte recovery in peripheral blood by direct measurement, although this is a primary clinical correlate.

### SUMMARY OF THE INVENTION

It has been discovered by the present inventors that a beta glucan composition increased the number of CD34+ precursor cells ex vivo in expansion culture using umbilical cord blood samples, and promoted homing and engraftment of CD34+ enriched cord blood cells in the NOD/SCID mouse in vivo. It has also been discovered in accordance with the invention that a beta glucan composition protected against bone marrow myelotoxicity caused by chemotherapy. Accordingly, the present invention provides methods based on the use of a beta glucan composition. Beta glucans suitable for use in the invention can be chemically synthesized or extracted from a variety of sources of organisms.

In one embodiment, this invention provides a method to expand CD34+ cells in an initial population of cells by culturing said population in vitro with a composition containing beta glucan.

In another embodiment, this invention provides a method to promote homing of an administered population of cells to the bone marrow of a mammal by culturing said population with a beta glucan composition in vitro prior to its administration.

In still another embodiment, this invention provides a method to promote engrafting of an administered population of cells to the bone marrow of a mammal by culturing said population with a beta glucan composition in vitro prior to its administration.

In a further embodiment, this invention provides a method to promote homing of an administered population of cells to the bone marrow of a mammal by administering a beta glucan composition to the mammal. This invention also provides a method to promote engrafting of an administered population of cells to the bone marrow of a mammal by administering a beta glucan to the mammal. The beta glucan composition may be administered orally or intermixed with the population of cells for administration via, e.g., a parenteral route. The beta glucan composition may be administered prior to, at the time of, or after administration of the population of cells.

In another embodiment, this invention provides a method to reduce the hematologic toxicity of chemotherapy associated with cancer treatment in a mammal by administering a beta glucan to the mammal. The beta glucan may be administered orally, and may be administered prior to, at the time of, or in the period following administration of a chemotherapeutic compound. The chemotherapeutic compound may be a taxane and does not include doxorubicin.

### BRIEF DESCRIPTION OF THE DRAWINGS

FIG. 1: Effect of MBG on Expansion of HPC and HSC in Cord Blood ex vivo. Panel A. Mononuclear cells were separated from cord blood from healthy infants (n=4) enriched for CD34+ cells and cultured ex vivo in the presence or absence of MBG at the indicated doses. The effects of MBG on expansion of cell populations were determined after 4 days of culture followed by harvesting, staining with anti-human CD34, CD38, CD33 antibodies, and assessment by three-color flow cytometry. Data in Panel A show the CD34+CD33+ CD38−(HPC) cell population. Data in Panel B show the CD34+CD38−CXCR4+ (HSC) cell population from the same cultures as in Panel A. Data present mean cell numbers ±SD, *p<0.05 vs. control with no MBG.

FIG. 2: Expression of Dectin 1 on Cord Blood Cells. CB samples were stained with dectin-1 antibody, using a modified indirect staining protocol to detect dectin-1, and directly conjugated fluorescent antibodies to CD45, CD19, and anti-CD14 were used for assessment of B cells and monocytes; all analyzed by three-color flow cytometry. Data in Panel A show flow cytometric histogram overlays for one term infant's monocyte and B cell populations. Data show the percentage of each respective gated population that expresses dectin-1 compared to the isotype control. Data in Panel B represent the mean percentage ±SD of monocytes and B cells in the CB group that express dectin-1. Samples were from 12 healthy full term infants.

FIG. 3: Effect of MBG on Homing of CB CD34+ cells in NOD/SCID Mice. Data show effect of daily oral MBG treatment at 4 mg/kg/day at 3 days after CB transplant compared to control group mice. Control 1(Ctr1) group mice (n=4) were transplanted with same CB as the MBG1 group of mice (n=4), while control 2 (Ctr2) mice (n=4) and MBG 2 mice (n−4) received the same other unit of CB. CD34+CD45+human CB cells were retrieved from bone marrow (A) and spleen (B) and analyzed by flow cytometry (** p<0.01 vs. control).

FIG. 4: Effects of MBG on Engraftment of CB CD34⁺ Cells in NOD/SCID Mice. The MBG group mice were given 4mg/kg/day of MBG beginning on the day of CB transplantation and during the subsequent 6 days. Control 1 (Ctr1) group of mice and MBG1 group mice were transplanted with the same unit of CB, while control 2 (Ctr2) group of mice and the MBG2 group of mice received the other same unit of CB. Human CD34+CD45+ cells retrieved from NOD/SCID mice bone marrow (A) or spleen (B) were analyzed by flow cytometry (**p<0.01 vs. control).

FIG. 5: Comparison of Response to MBG after Transplantation in NOD/SCID Mice. The MBG group mice were given 4 mg/kg/day of MBG on the day of transplantation and over the subsequent 3 or 6 days (n=8, n=8, respectively). Human CD34+CD45+ cells retrieved from NOD/SCID mice bone marrow (Panel A) or spleen (Panel B) were analyzed by flow cytometry. Data show mean percentage±SD, **p<0.01 vs. control.

FIG. 6: Effect of MBG on MCF-7. Panel A shows the effect of MBG on MCF-7 cell viability as determined by the MTT test after treatment with MBG at the indicated doses for 72 hrs. Panel B shows the effect of paclitaxel on MCF-7 viability in the presence and absence of MBG at 100 micrograms/ml.

FIG. 7: Paclitaxel Induction of Leukopenic Mouse Model. Ptx was given at cumulative doses of 60 mg/kg and 90 mg/kg. Complete blood counts (CBCs) were compared to untreated mice (n=4 each group) over 10 days. Comparison of mean absolute counts of leukocytes, neutrophils, and lymphocytes are shown. The arrows on the top in each chart indicate days of Ptx injection. Data are shown as group mean absolute counts±SD.

FIG. 8: MBG Enhancement of CFU-GM Activity after Ptx. Two days after the last Ptx injection, bone marrow (BM) and spleen (SP) cells were collected for ex vivo colony forming unit assays (CFU). (A.) Ptx+MBG 4 mg/kg/day treated mice had significantly higher CFU-GM counts in BM and SP (p<0.001, p=0.002, respectively) compared to Ptx alone by ANOVA. (B.) Ptx+MBG 6 mg/kg/day led to increased CFU-GM in BM (p−0.003), and showed a trend towards increase in SP.

FIG. 9: Effect of MBG on Leukocyte Recovery after Ptx. (A.) The decrease in white blood cell (WBC) count was less in the Ptx+MBG group compared to Ptx-alone (p=0.024), or after Ptx+G-CSF (p−0.031). (B.) At 2 days post Ptx, the decline WBC in the Ptx+MBG group was less compared to Ptx-alone (p<0.05), or G-CSF (p<0.01) since the effect of G-CSF was not evident 24 hrs after injection. (C.) On day 8 post Ptx, both Ptx+MBG and Ptx+G-CSF groups had similar mean WBC counts, that were higher compared to Ptx-alone (p<0.001).

FIG. 10: Effect of MBG on Neutrophil Recovery After Ptx. (A.) Ptx decreased neutrophil counts in all groups that lasted until post Ptx day 5 for the Ptx+MBG and Ptx+G-CSF groups. (B.) On post Ptx day 5, neutrophils were less decreased in the Ptx+MBG (p<0.05) and Ptx+G-CSF (p<0.01) groups compared to Ptx alone. (C.) On day 8 post Ptx, neutrophil counts had rebounded far above baseline after both Ptx+MBG and Ptx+G-CSF but not after Ptx-alone.

FIG. 11: Effect of MBG on Lymphocyte Recovery after Ptx. Ptx reduced lymphocyte numbers in all groups compared to the baseline (p<0.0001). By post day 5, lymphocyte counts were higher than baseline for the Ptx+MBG (p<0.01) but not the Ptx+G-CSF group. On post day 8 counts were higher than baseline for both Ptx+MBG and Ptx+G-CSF groups (p<0.01) but not for Ptx-alone.

FIG. 12: MBG Promoted Early Recovery of Myeloid Function. (A.) Peripheral blood Gr-1+ granulocyte/monocyte production of reactive oxygen species (ROS) was tested ex vivo 4 days after Ptx by flow cytometry. Response to E. coli in the Ptx-alone and Ptx+G-CSF groups was lower compared to untreated mice (p<0.01 for both). ROS response was higher in the Ptx+MBG group compared to Ptx alone (p<0.01) or Ptx+G-CSF (p<0.01), and equal to untreated mice. Response to fMLP stimulation was higher in the Ptx+MBG group than either untreated mice (p<0.01) or the Ptx+G-CSF group (p<0.05). (B.) At post Ptx day 11 ROS response to E. coli was now equal in all treated groups and higher than in untreated mice (p<0.0001). However, fMLP response in the Ptx+MBG group was higher compared to Ptx-alone or Ptx+G-CSF groups (p=0.013 and p=0.014, respectively).

### DETAILED DESCRIPTION OF THE INVENTION

The present invention is predicated at least in part on the discovery that a beta glucan composition (such as maitake beta glucan extract, or “MBG”) increased the number of CD34+ precursor cells ex vivo in expansion culture using umbilical cord blood samples, and promoted homing and engraftment of CD34+ enriched cord blood cells in the NOD/SCID mouse in vivo. Without being bound to any specific theory, it is likely that the effects of MBG on the expansion of hematopoietic progenitor cells ex vivo are mediated by the CD33+ monocyte population, previously shown to produce G-CSF in response to beta glucan. Additionally, without being bound to any specific theory, it is likely that the effect of MBG on human precursor cell engraftment in the mouse involves parallel effects on mouse bone stromal macrophages and/or other indirect mechanisms such as support of mouse bone marrow recovery such as increasing expression of stromal cell-derived factor (SDF)-1 alpha. The discovery of the invention is significant for the development of progenitor cells from cord blood, which are useful as a resource for clinical transplantation. Based on the discovery, the present invention provides methods based on the use of a beta glucan composition to expand CD34+ cells in an initial population of cells in a culture in vitro, and to promote homing and engraftment of a cell population containing CD34+ cells to the bone marrow of a recipient.

It has also been discovered in accordance with the invention that a beta glucan composition (such as maitake beta glucan extract, or “MBG”) protected against bone marrow myelotoxicity caused by chemotherapy. Specifically, in examining the effects of MBG on leukocyte recovery and granulocyte/monocyte function in vivo after dose intensive paclitaxel (Ptx) in a normal mouse, it has been discovered that leukocyte counts declined less in mice treated with Ptx and MBG compared to Ptx-alone or Ptx+G-CSF treatment; that lymphocyte levels were higher after Ptx+MBG but not Ptx+G-CSF treatment compared to Ptx alone; that MBG increased CFU-GM activity in bone marrow and spleen two days after Ptx; that on day 4 post-Ptx MBG restored granulocyte/monocyte ROS response to normal levels as compared to Ptx-alone and increased ROS response compared to Ptx-alone or Ptx+G-CSF. Accordingly, the present invention further provides methods based on the use of a beta glucan composition to reduce hematologic toxicity of chemotherapy associated with cancer treatment.

### Beta Glucan Compositions

Beta glucans are most frequently found in cell walls of bacteria, fungi (including yeast and mushrooms such as Reishei, Shiitake and Maitake), seaweed and grains (such as oats, barley, rye and wheat).

Beta glucans are polysaccharides of D-glucose monomers linked by beta glycosidic bonds. The types of beta-linkages in a particular beta glucan can include beta (1, 3), beta (1, 4), beta (1, 6), or a combination thereof. One example of a beta glucan suitable for use in the invention is a beta-glucan containing D-glucose units attached to one another at the (1, 3) position (i.e., main chain) with side chains of D-glucose attached at the (1, 6) position. Another example is a beta-glucan containing D-glucose units attached to one another at the (1, 6) position (i.e., main chain) with side chains of D-glucose attached at the (1, 3) position.

Suitable sources of beta glucans include beta (1, 3)D glucan derived from the cell wall of Saccharomyces cerevisiae, beta-(1, 3)(1, 4) glucans extracted from the bran of grains such as oats and barley, PGG-glucan (poly-1-6 beta-D-glucopyranosyl 1,3-beta-glucopyranose) derived from yeast (such as Saccharomyces cerevisiae) or from mushrooms such as Grifola frondosa, Sclerotinia sclerotiorum and Sparassis crispa (8-12), SSG (a 1,3-beta-D-glucan) obtained from the culture filtrate of S. sclerotiorum (9); and SCG, a 1,3-beta-D-glucan from Sparassis crispa (7, 9, 15).

By “a beta glucan composition”, “a beta glucan extract” or “a beta glucan preparation” is meant a composition, extract or preparation that contains a beta glucan or beta glucans as the principal component(s) of the composition or preparation; i.e., the beta glucans account for at least 50% w/w of all components, or at least 60%, 70%, 80%, 85%, 90%, 95%, 98%, 99% w/w or greater (including 100%), or a percentage falling within a range fainted by any two of these values. Other minor components that may be present in a beta glucan composition include, for example, proteins, lipids, nucleic acids, or other organic or inorganic compounds, which may be in complex with (associated with) the beta glucan molecules in the preparation.

Beta glucans can be chemically synthesized, or extracted from a variety of sources of organisms identified above based on protocols and techniques well documented in the art. An example of a beta glucan composition for use in the invention is an extract prepared from the fruit bodies of a Maitake mushroom (Grifola), also referred hereinbelow as “MBG”. MBG can be extracted according to the methods described in U.S. Pat. No. 5,854,404, for example.

Beta glucan compositions or preparations can be in different physical forms initially, including both soluble and particulate (i.e., non soluble particles) forms. A beta glucan preparation can be optionally combined with one or more other active agents, carriers or diluents in an appropriate mariner for use either in vitro or in vivo. The other components that can be combined with a beta glucan preparation may depend on the manner in which the composition is to be administered. For example, a beta glucan extract can be combined with a filler (e.g., lactose), a binder (e.g., carboxymethyl cellulose, gum arabic, gelatin), an adjuvant, a flavoring agent, a coloring agent and a coating material (e.g., wax or plasticizer) to formulate a composition in tablet or capsule foam for oral administration. A beta glucan extract can be combined with an emulsifying agent, a flavoring agent and/or a coloring agent to formulate a composition in liquid form suitable for ingestion or injection. A beta glucan extract can be combined with, dissolved or emulsified in water, sterile saline, phosphate buffered saline (PBS), dextrose or other biologically acceptable carrier, for parenteral administration.

Expansion of CD34+ Cells from an Initial Cell or Sample Source

In one embodiment, the present invention provides a method for expanding CD34+ cells in an initial population of cells by culturing the initial population of cells in vitro in the presence of a beta glucan composition to expand in a culture.

The initial population of cells refers to a population of cells obtained or obtainable from a sample source including, for example, bone marrow, peripheral blood, umbilical cord blood spleen or other tissues such as dental pulp, which contains human hematopoietic progenitor cells, including lineage-restricted and committed progenitors (also referred to herein collectively as “HPC”), and/or primitive uncommitted hematopoietic stem cells (“HSC”) that sustain multilineage hematopoiesis. Other appropriate sample sources can be used, including modified cells (e.g., genetically modified cells), and hematopoietic stem and/or progenitor cells developed in vitro using embryonic or adult stem cells. In a specific embodiment, umbilical cord blood of a mammal, e.g., a human subject, is used as the source to obtain the initial cell population.

An appropriate sample source can be processed to obtain and extract an initial cell population containing hematopoietic stem cells, and optionally an initial cell population also containing and optionally enriched with hematopoietic progenitor cells. Various commercial kits are available for extracting and enriching hematopoietic stem/progenitor cells from a blood sample or any other source of stem/progenitor cells, e.g., the RosetteSep cord blood stem/progenitor enrichment system from StemCell Technologies Inc. (Vancouver, Canada).

According to the invention, the initial population of cells is then cultured in vitro in an appropriate culture medium in the presence of a beta glucan composition to expand CD34+ hematopoietic progenitor cells in the cell population.

By “expanding” is meant that the number of CD34+ hematopoietic progenitor cells in the cell population is increased as a result of the culture with a beta glucan composition, as compared to culturing in the absence of the beta glucan composition. The increase should be at least significant as determined by any one of art-recognized statistic method, and is preferably at least 20%, 30%, 40%, 50%, 60%, 70%, 80%, 90%, 100%, 125%, 150%, 175%, 200% or greater.

In one embodiment, the culture in the presence of a beta glucan composition results in an expansion of a subset of CD34+ hematopoietic progenitor and/or stem cells, wherein the subset is identified based of CD33 or other comparable marker or CXCR4 and has low or absent expression of CD38 in vitro and expresses CD45+, all of which are markers documented in the art. In a specific embodiment, the culture results in an expansion of CD34+CD38-(or dim) cells. In another specific embodiment, the culture results in an expansion of CD34+CD33+CD38-cells. In still another embodiment, the culture results in an expansion of CD34+CD38-CXCR4+cells.

The culture medium suitable for use in the invention includes any standard, conventional culture medium suitable for culturing and maintaining stem cells or progenitor cells, such as hematopoietic stem or progenitor cells, supplemented with a beta glucan composition. The culture medium can include additional growth factors and cytokines such as Flt-3, SCF, IL-3, IL-5, among others, or a combination thereof.

The amount of a beta glucan composition in the culture medium is effective to enrich the CD34+ cells in the cell population, and may depend on the specific composition and beta glucan. Generally speaking, the amount of a beta glucan composition in the culture medium may be in the range of 10 to 500 μg/ml, or 25-400 μg/ml. In specific embodiments, the amount is at least 25, 50, 75, 80, 90, 100, 110, 120, 130, 140, 150, 160, 175, 200, 225, 250, 275, or 300 μg/ml; and in other specific embodiments, the amount is not more than 300 μg/ml, or not more than 250, 200, 175, 150 or 125 μg/ml. The precise amount of a specific beta glucan composition can be determined by one skilled in the art based on the disclosure of the invention. For example, it has been determined that MBG is especially effective at an amount of 50 μg/ml or 100 μg/ml.

The resulting cell population after culturing with a beta glucan composition, now enriched with CD34+ hematopoietic progenitor and/or stem cells, can be transplanted to a subject, or returned to the subject who provided the initial cell population (i.e., the donor). It has been found in accordance with the invention that an initial cell population containing hematopoietic progenitor and/or stem cells to be administered to a subject for transplantation, if treated (e.g., culturing as described above) in vitro with a beta glucan composition, is improved in its homing and engraftment to the bone marrow of the recipient.

Accordingly, in one embodiment, the invention provides a method of promoting homing of a cell population containing hematopietic progenitor and/or stem cells to the bone marrow of a recipient by treating (e.g., culturing) the cell population in vitro with a beta glucan prior to administration of the cell population to the recipient. In another embodiment, the invention provides a method of promoting engrafting of a cell population containing hematopietic progenitor and/or stem cells to the bone marrow of a recipient by treating (e.g., culturing) the cell population with a beta glucan prior to administration of the cell population to the recipient.

By “homing” to the bone marrow refers to the ability of the transplanted cells to find their way to locate to bone marrow of the recipient. The extent of homing can be determined by retrieving bone marrow from a transplantation recipient shortly after transplantation (e.g., within 1, 2, 3, 4, or 5 days of transplantation) and determining the number of transplanted cells found in the bone marrow.

By “engraftment” to the bone marrow refers to the ability of the transplanted cells to integrate or insert into the bone marrow of the recipient. The extent of engraftment can be determined by retrieving bone marrow from a transplantation recipient after transplantation, generally after at least 5 days, or 6, 7, 8, 9, 10, 15, 20, 30 days or longer after transplantation, and determining the number of transplanted cells found in the bone marrow.

By “promoting” or “enhancing” homing or engraftment refers to an increased number of transplanted cells in the bone marrow of a recipient. The increase should be at least significant as determined by any one of art-recognized statistic method, and is preferably at least 20%, 30%, 40%, 50%, 60%, 70%, 80%, 90%, 100%, 125%, 150%, 175%, 200% or greater.

### Administration of a Beta Glucan Composition to a Transplantation Subject

Further in accordance with the present invention, beta glucan compositions can be administered directly to a transplantation recipient (including human subjects) in order to improve homing and engraftment to the bone marrow of a transplanted cell population containing hematopoietic progenitor and/or stem cells. In certain specific embodiments, homing and engraftment of CD34+ hematopoietic progenitors and/or stem cells to the bone marrow are achieved and enhanced by administering a beta glucan composition to the recipient.

A beta glucan composition can be administered to a recipient of transplantation before (one or several days before or immediately before), simultaneously with, and/or after (one or several days or one or several weeks) transplantation. The frequency of administration can be one or more times per day every day, every other day, or every 2-3 days. The precise amount given to an individual may depend on the conditions (e.g., health and weight) of the recipient, and the specific beta glucan composition. Generally speaking, for human subjects, the amount of a beta glucan composition suitable for administration can be in the range of 4-12 mg/kg/day or higher.

A beta glucan composition can be administered via any appropriate route, including oral or parenteral route.

### Use of a Beta Glucan Composition in Chemotherapeutic Cancer Therapy

In a further embodiment, the present invention provides methods of reducing hematologic toxicity of chemotherapy associated with cancer treatment based on use of a beta glucan composition.

Chemotherapy used cancer treatment, especially treatment of aggressive forms of cancer, is commonly associated with toxicity to the hematologic system, including bone marrow suppression. Chemotherapeutic compounds often kill fast-growing blood-forming cells by damaging bone marrow and hematopoietic progenitor and stem cells, the source of blood cells, as well as cancer cells. Hence, hematologic toxicity caused by chemotherapy manifests as neutropenia or leukopenia (loss of the myeloid lineage or lymphoid lineage). Neutrophil development is especially sensitive to chemotherapy. The most common form of clinical hematotoxicity is shown by low numbers of infection-fighting neutrophils (neutropenia) but also involves monocytes and frequently lymphocytes (known collectively as leukocytes or white blood cells) and this is shown by a reduced white blood cell count in peripheral blood. The hematotoxicity of chemotherapy may also include anemia (loss of the erythroid lineage, low red blood cell count), and thrombocytopenia (low platelet count), or combinations thereof.

As used herein, cells of the hematologic system include bone marrow hematopoietic cells (including pluripotent hematopoietic stem cells, committed and lineage-restricted progenitor cells, mesenchymal stem cells, and stromal cell and other cells required for production of normal blood in bone marrow or spleen), red blood cells (including erythroid precursor, and white blood cells or leukocytes including basophils, eosinophils, neutrophils which comprise granulocytes, as well as monocytes, mast cells, macrophages, lymphocytes such as T and B lymphocytes and NK, NK-T, and dendritic cells).

The toxic effects of a chemotherapeutic compound on cells of the hematologic system of a patient can be evaluated by a range of assays, including complete blood cell count, white blood cell count, speed and/or extent of recovery for red or white blood cells, various types of colony forming assays including colony forming unit-granulocyte/monocyte (CFU-GM) activities in bone marrow, spleen or peripheral blood (mobilization), G-CSF production, and functional assays such as granulocyte/monocyte production of reactive oxygen species (ROS) in response to specific and non specific signals.

It has been discovered by the present inventors that a beta glucan composition protects against hematologic toxicity caused by chemotherapy. Chemotherapy is typically given periodically in multiple cycles continuing for many weeks. The spacing between cycles provides opportunity for patients to recover the damaged hematologic system, and renew cell populations. Since this spacing may also allow recovery of cancer cells or cancer stem cells, dose intensified treatment aimed at cancer destruction is now widely used. However, the treatment often has to be interrupted, reduced, or discontinued as a result of toxicity. A treatment regimen that incorporates administration of a beta glucan composition protects and promotes recovery of the cells of the hematologic system, and therefore permits timely completion of chemotherapy under circumstances that otherwise would have been difficult or impossible, or even allows therapy with the same chemotherapeutic compound at an elevated dose.

By “reducing hematologic toxicity” is meant the toxic effect, measurable in any one of the above assays of hematologic cells or cell functions caused by chemotherapy, is reduced as compared to chemotherapy absent the beta glucan composition. The reduction should be statistically significant.

A suitable beta glucan composition can be incorporated in chemotherapies based on any chemotherapeutic anti-cancer drug or a combination of drugs, especially those that cause significant hematologic toxicity. Examples of chemotherapeutic drugs include Carboplatin, Cetuximab, Cisplatin, Cyclophosphamide, Paclitaxel, Docetaxel (Taxotere), and Trastuzumab, and are by no means limiting the scope of the present invention.

Functional derivatives of a drug, i.e., derivatives that maintains the desired pharmacological effect of the drug, can also be used in practicing the present invention, such as salts, esters, amides, prodrugs, active metabolites, analogs and the like. Chemotherapeutic drugs contemplated by the invention do not include doxorubicin. The exact dose, timing and route of the administration of a chemotherapeutic drug is documented in the art, or can be determined by the treating physician using standard procedures.

A beta glucan composition can be administered to a cancer patient before, during, or after the administration of a chemotherapeutic compound. The frequency of administration can be one or more times per day every day, every other day, or every 2-3 days. The precise amount given to an individual may depend on the conditions (e.g., health and weight) of the recipient, and the specific beta glucan composition. Generally speaking, the dosage amount of a beta glucan suitable for administration can be in the range of 4 to 12 mg/kg/day or higher. A beta glucan composition can be administered via any appropriate route, including oral or parenteral route.

The methods of the present invention are applicable for treating a range of cancers, particularly solid tumors, including but are not limited to breast, esophagus, nasopharynx, colon, pancreas, cecum, lung, and prostate cancer myeloid deficiency states such as myelodysplastic syndromes.

The experiments described in Examples 1-5 were conducted to determine the effects of MBG on the proliferation and differentiation of phenotypically distinct subpopulations of cord blood (CB) progenitor and stem cells during expansion of freshly obtained CB from healthy full term infants cultured ex vivo and to evaluate the potential role of oral administration of MBG on the fate of CB CD34+ precursor cells in vivo in the NOD/SCID mouse model for homing and engraftment.

### EXAMPLE 1

### Material and Methods:

Chemicals and Reagents: Maitake mushroom beta-glucan (MBG) used in the following examples is an extract from fruit body of Maitake mushroom (Grifola frondosa), which was made according to the methods described in U.S. Pat. No. 5,854,404 (corresponding to Japan Patent No. 2859843), and was provided by Yuikiguni Maitake Corp. through the Tradeworks group. The extract was stored in a refrigerator at 4° C. under dark conditions until use. The lot of MBG used in this study was sent to NAMSA to test for endotoxin contamination using limulus amebocyte lysate (LAL) assay. The result showed that there was no detectable endotoxin activity (maximum level=0.012 EU/mg). MBG powder dissolved readily in RPMI 1640 with 25 mM HEPES buffer and was initially prepared at a concentration of 20 mg/ml and sterilized by filtration through 0.2 μm cellulose acetate low protein binding membrane, and stored at −20° C. The stock solution was diluted to the required concentration in RPMI 1640 medium freshly at the time of use.

Mice: NOD.CB17-Prikdc scid/J mice were purchased from Jackson Laboratory, and maintained under a restricted barrier facility at Memorial Sloan-Kettering Cancer Center (MSKCC, New York, N.Y.). All animal experiments were approved by the Animal Care Committee of MSKCC. Mice were maintained on regular food, Certified Rodent Diet # 5053 (LabDiet) throughout the study. Mice, 8-10 weeks old, were given a sub-lethal dose (350 cGy) of whole body irradiation at a rate of 65 cGy/min from a Gammacell 40 Exactor containing ¹³⁷Cs (MDS Nordion; Kanata, Ontario Canada). Within 24 hrs, mice were injected through the tail vein with CD34+ enriched human umbilical cord blood cells (2-5×10⁵cells/mouse). Mice were sacrificed using the CO₂ technique at different time points after transplantation as indicated. Mouse peripheral blood was obtained by cardiac puncture bleeding at the time of sacrifice and mouse bone marrow and spleen cells were collected and resuspended as single-cell suspensions.

Human Cord Blood Samples: Human umbilical cord blood (CB) samples from healthy full term infants were obtained under an approved IRB protocol at Weill Medical College of Cornell University. All CB units used in this study were released by the New York Blood Bank Cord Blood Banking program at New York Presbyterian Hospital—Weill Cornell due to low volume or for logistical reasons. All samples were collected at the time of delivery into blood bags containing anticoagulant Citrate-Phosphate Dextrose Adenine and processed freshly in the Weill Cornell Cellular Immunology Laboratory.

Cord Blood Cell Dectin-1 Expression: Freshly collected CB samples were stained with mouse anti-human dectin-1/CLEC7A antibody (R&D systems, Minneapolis, Minn.) using a modified indirect staining protocol. Briefly, blood samples were lysed with BD Pharm Lyse TM for 10 min in room temperature (RT) to remove red blood cells, after washing with staining buffer (PBS/0.5% BSA) twice, cells were incubated with 400 uL blocking buffer 1 containing 0.5% human IgG, 5% BSA, 2 mM NaN3 in PBS at 4° C. for 20 min to block against human Ig Fe receptor (FcR). Cells were then washed with 2 mL staining buffer once, followed by staining with mouse anti-human dectin-1/CLEC7A antibodies or matching isotype antibodies for 30 min at 4° C. After washing with 2 mL staining buffer once, cells were incubated with secondary antibodies, FITC conjugated goat anti-mouse IgG (R&D systems, Minneapolis, Minn.) at 4° C. for 30 mins, then washed with 2 mL staining buffer. Afterwards, cells were incubated with 0.5 mL of blocking buffer II (5% mouse serum in PBS) for 20 mins at 4° C., and washed once with 2 mL staining buffer. Directly conjugated fluorescent antibodies of interest were then added (e.g. CD14 PE for detecting monocytes, CD45 PerCP and CD19 PE for detecting lymphocytes), and samples were incubated at R.T for 15 mins, then washed with 2 mL staining buffer. Cells were resuspended in 400 uL fixative solution containing 1% paraformaldehyde, 0.25% BSA, 1 mM NaN3 in PBS. The cells were then acquired and analyzed in a FACSCalibur flow cytometer (BD) using Cell Quest and FlowJo software. Gating was initially performed on monocytes by light scatter properties and on lymphocytes using anti CD45. The anti-dectin-1 antibody GE2 (IgG1) provided by J A Willament was used as a reference.

Cord Blood Stem Cell Enrichment: Freshly collected CB samples were enriched for CD34⁺ stem cells using the RosetteSep cord blood progenitor enrichment system (StemCell Technologies Inc. Vancouver, Canada) according to the manufacturer's instructions. Briefly, RosetteSep human progenitor enrichment cocktail was added into CB at 50 u1/ml blood, incubated at room temperature (RT) for 20 mins. After incubation, the CB was diluted with PBS/2% FBS at 1:4 (v/v), and mixed well. The diluted blood was layered on top of Ficoll-Paque. After centrifugation for 25 min at 2000 rpm at RT, the enriched cells were collected from the Ficoll-Paque plasma interface. Cells were washed with PBS/2% FBS twice, and then resuspended in PBS/2% FBS. Cell aliquots were diluted and mixed using Turk's stain and counted by light microscope using a hemocytometer. For each CB sample, a small aliquot of enriched cells was assessed by flow cytometric technique to detect the percentage of enriched CD34+CD33+CD38− cells as well as CD34+CXCR4+CD38− cells; the mean percentages for the CB samples used in ex vivo expansion studies were 8.2±10.0 and 2.8+1.9, respectively.

Ex Vivo Expansion Assay: Cord blood was enriched for CD34+ progenitor cells using RosetteSep cord blood progenitor enrichment system (StemCell Technologies Inc. Vancouver, Canada), after separation of mononuclear cells by density gradient centrifugation as described above. CD34-enriched cord blood cells were then cultured in expansion culture medium, which was StemSpan® H3000 medium (StemCell Technologies Inc.) with StemSpan™ CC100 cytokine cocktail containing 100 ng/mL rh Flt-3 ligand, 100 ng/mL rh Stem Cell Factor (SCF), 20 ng/mL rh interleukin-3 (IL-3) and 20 ng/mL rh interleukin-6 (IL-6). Briefly, after washing with PBS/12% FBS, CD34-enriched cord blood cells were resuspended in expansion medium at 6.69±1.74×10⁴ cells/mL. After adding MBG (final concentrations were 0, 50, 100, 200 μg/ml), CD34+ enriched cells were cultured in expansion culture medium at a total volume 2 mL in T25 tissue culture flasks. The cells were cultured at 37° C., in a 5% CO₂ humidified incubator.

Harvesting and evaluation of cell populations in cell cultures was performed at specific time points: 0, 4, 7, and 14 days. Effects of MBG on expansion of cell populations with either CD34+CD33+CD38−HPC or CD34+CD38−CXCR4+ HSC phenotypes were assessed by flow cytometry. Flow cytometric analysis was performed on cells stained with directly conjugated moAb using a FACSCalibur (BD Biosciences) instrument. Stepwise gating was performed first to gate on CD38-mononuclear cells expressing CD34 then to determine percentage of populations co-expressing CD33 (HPC) or co-expressing CXCR4 by three-color flow cytometry. Data acquisition and analysis were performed with CellQuest and FlowJo software.

Labeling of CD34+ Enriched Cord Blood Cells with CFSE: For the homing studies, CD34+ enriched cord blood cells were labeled with carboxyfluorescein diacetate succinimidyl ester (CFSE) 18 hrs before injection into NOD/SCID mice. Briefly the CFSE solution was added to enriched CB cell suspensions, and samples were incubated at 37° C. for 15 mins. Then pre-chilled (4° C.) PBS/0.1% BSA was added to wash the cells. Aspiration was performed and cells were washed twice more with pre-chilled PBS/0.1% BSA. After the final aspiration, 5 mL of pre-warmed (37° C.) RPMI-1640/5% FBS was added and cells were put into a 37° C., 5% CO₂ incubator overnight.

Injection of CD34+ Enriched Cord Blood Cells into NOD/SCID Mice: Before injection, the cells were washed with PBS once and resuspended in PBS at 1˜2.5×10⁶ cells/ml. Injection of 200 uL/mouse of the enriched CB cells was performed through the tail vein into NOD/SCID mouse which had been given sublethal irradiation on the previous day.

MBG Oral Administration: In the MBG treatment group, mice were orally given 4 mg/kg.day of MBG by gavage at the same time as the transplantation and then were given MBG daily in the subsequent experimental days in the same way. The mice were weighed each day before gavage.

### Collection of Mouse Peripheral Blood, Bone Marrow and Spleen:

Peripheral Blood: After experiment period, mice were sacrificed with the CO₂ technique. Mouse peripheral blood was obtained by cardiac puncture allowing free flow bleeding into small, heparinized sterile tubes.

Bone Marrow: Mouse bone marrow cells were collected by standard procedure as previously described (8). Briefly, mouse bone marrow cells were collected from femoral shafts by flushing with 3 mL of cold RPMI-1640. The cell suspensions were passed up and down six times through an 18-gauge needle in RPMI-1640 to disperse cell clumps. After washing once with RPMI-1640, bone marrow cells were incubated with 15% FBS/RPMI-1640 at RT for 30 mins. After washing with serum-free RPMI-1640 twice, the cells were washed once with PBS, and resuspended in PBS for staining with fluorescent conjugated monoclonal antibodies.

Spleen: Mouse spleen cells were collected with smearing between two sterile glass-slides a few times in˜3 mL RPMI-1604 medium. The cell suspensions were passed up and down through an 18-gauge needle in RPMI-1640 to disperse cell clumps. After washing with RPMI-1640 once, mouse spleen cells were incubated with 15% FBS/RPMI-1640 at RT for 30 mins. Spleen cells were washed twice with serum-free RPMI-1640, then washed once with PBS, and then resuspended in PBS for staining with fluorescent-conjugated monoclonal antibodies.

Staining with Anti-Human CD45 and CD34 Antibodies to Detect Engrafted Human Cells: After washing, bone marrow, or spleen cells with PBS, peripheral whole mouse blood, bone marrow or spleen cells were pre-incubated with Mouse BD Fc Block (purified anti-mouse CD16/CD32 mAB, 2.4G2, BD Biosciences) at≦1 ug/million cells in 100 uL, at 4° C. for 5-10 mins. Then monoclonal antibodies of interest were added: mouse IgG R-PE and mouse IgG- PerCP for isotype detection tubes, anti-human CD45-PerCP and anti-human CD34-PE for the human cell detection tubes. After incubating at RT for 15 mins in the dark in the presence of Mouse BD Fc Block fixative-free lysing solution was added at 2 mL/tube (High-Yield Lyse, CALTAG Carlsbad, Calif.), followed by vortexing and incubation at RT in dark conditions for 10 mins. Then tubes were centrifuged at 1500 rpm for 5 mins, and vacuum aspirated to remove supernatants. After washing the cells once with PBS, followed by aspiration to remove supernatants, 7-AAD was added and tubes were incubated at 4° C. for 15 mins. Then cells were treated with 0.5 ml of fixative solution (7.5 g paraformaldehyde +2.5 mL FBS in 500 mL of PBS). The cells were then acquired and analyzed in a FACSCalibur flow cytometer (BD) using Cell Quest and FlowJo software.

Flow Cytometric Analysis: Flow cytometric analysis was used to determine the percentage and number of human CD45 and CD34 cells retrieved from the NOD/SCID mouse bone marrow, spleen and peripheral blood. For homing studies, human CB cells were identified first with CFSE labeling, then CD34 R-PE or CD45 PerCP positive cells were gated. For engraftment studies, the gating strategy was performed as described (34). Dead cells were excluded using 7-AAD by plotting 7-AAD against forward light scatter. Living CD34 R-PE and/or CD45-FITC positive cells were then gated. To determine the number of CD34+ CB cells retrieved from the NOD/SCID mouse 6 days after transplantation, dead cells were excluded first using 7-AAD. Living CD45 dim cells possessing large forward light scatter properties were then gated and then plotted using CD45 FITC versus CD34 R-PE. The number of these cells represented the CD34⁺ CB cells retrieved from the NOD/SCID mouse bone marrow.

Statistical Analysis: Data are presented as mean percentage±SD or mean ±SD. To study the effects of MBG on the ex vivo expansion of CD34+ cells, one way ANOVA was used to examine the difference in average cell counts across different treatment groups. Dunnet's test was then used to compare the average cell counts between each of the MBG treated group and the control while properly adjusting for multiple comparisons. To further examine the differential treatment effects on HPCs and HSCs, two-way ANOVA with an interaction term of cell type and treatment was used. For the homing and engraftment studies, two-way ANOVA was used to examine the association between CD34+ cell homing and engraftment and MBG treatment while controlling for different cord blood samples transplanted. These analyses were carried out using statistical programming and software package R (35).

### EXAMPLE 2

### Effects of MBG on Expansion of CD34+ Cells Ex Vivo.

Umbilical cord blood samples were collected at delivery from healthy infants and processed within 12 hours. CD34+ progenitor cells were enriched using the RosetteSep human progenitor enrichment cocktail. Mononuclear cells were isolated by density gradient centrifugation, washed and evaluated for CD34⁺ cells by flow cytometry and then expanded ex vivo in StemSpan H3000 defined medium supplemented with growth factors and cytokines: rhFlt-3, rhSCF, rhIL-3, rhIL-6, in the presence or absence of MBG at various doses as indicated. The objective of these experiments was to assess the effects of MBG on expansion of the committed CD34+ progenitor cell expressing CD33+ an early marker of myeloid maturation as a correlate of potential HPC progenitor cell activity and on CD34+CXCR4+CD38− cells, putative HSC stem cells, as a correlate of uncommitted hematopoietic potential (36, 37). Absence of CD38 on CD34+ precursor cells was used to define the initial gate (37-39).

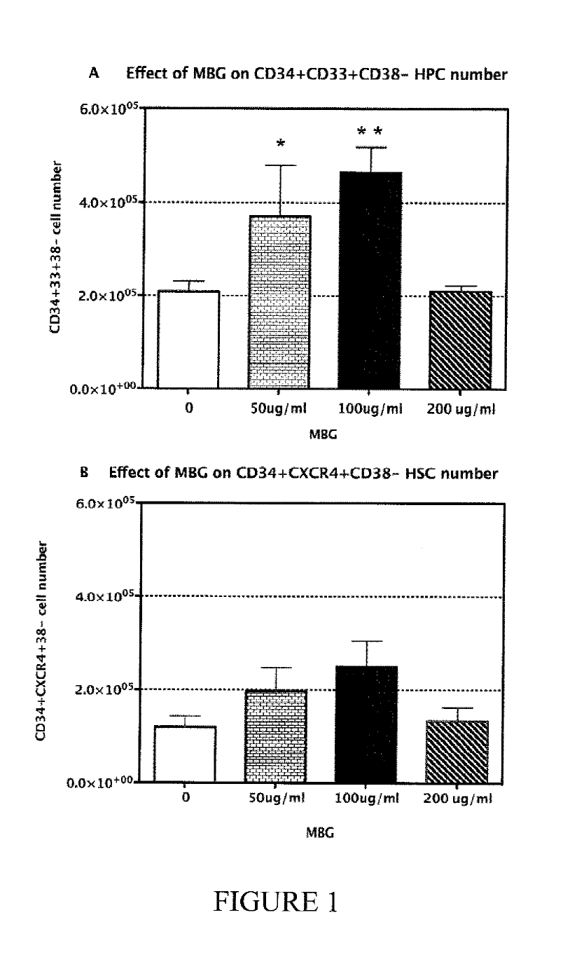

In the absence of cytokines and growth factors, CB cells did not proliferate and cultures were poorly viable (data not shown). After 4 days' culture in conventional expansion media with and without added MBG, expression of CD34, CD33, CD38 and CXCR4 was assessed by flow cytometry. After gating on CD34+CD38-cells, expression of CD33+ was assessed. Mean data from 4 experiments with CB from 4 different infants are shown in FIG. 1 panel A and panel B. As shown in panel A, MBG elicited a dose related enhancement of CD34+CD33+CD38-cells. Significant differences were observed using one-way ANOVA to analyze changes in HPC across all doses of MBG compared to conventional expansion medium for this population (p=0.002). Dunnet's test was then applied to evaluate pair-wise differences for specific doses of MBG. Significant increases in HCP were observed when MBG was added at 50 μg/ml and 100 μg/ml (p=0.022 and 0.003, respectively); expansion was maximal at 100 μg/ml. At 200 μg/mL the MBG response declined to the level of control cultures. The fall off was not due to any cytostatic or cytotoxic effects as determined in separate experiments adding MBG at 200 μg/ml to CB cells in the 2,3-bis (2-methoxy-4-nitro-5-sulfophenyl)-5-[(phenylamine) carbonyl]-2H-tetrazolium hydroxide (XTT) cytotoxicity assay. This could be due to cross-linking of the 1.3 branches at high concentration and failure to trigger monocyte activation.

The same samples from the same cultures were also assessed for expansion of precursor cells defined by expression of the CD34+CD38−CXCR4+ phenotype, which correlates with early, uncommitted hematopoietic stem cells (HSC) capable of repopulating the NOD/SCID mouse (46). Compared to conventional expansion medium alone, MBG treatment led to an increase in cells with HSC phenotype but these differences were not statistically significant by one way ANOVA. Data are shown in FIG. 1 panel B. As noted in Methods mean concentration of the inoculum was 6.69±1.74×104 cells /mL allowing comparison of all MBG doses and time points across all samples. There was no discernible impact of this difference on the results of the expansion studies.

In all samples there were more HPC cells than HSC cells. After enrichment the percentage of HPC cells was 8.2%±10.0 and that of HSC cells was 2.8%±1.9 as mentioned in the Methods section. The starting population of HSC cells was lower than that of HPC cells and could have influenced the overall expansion. To further examine whether there was a difference between the effect of MBG on HPCs and HSCs on ex vivo expansion in responding to MBG, a two-way ANOVA with an interactive term of cell type and treatment was used. The analysis showed that the HPCs showed a trend towards greater expansion compared to cells with HSC phenotype in response to MBG at 100 μg/ml.

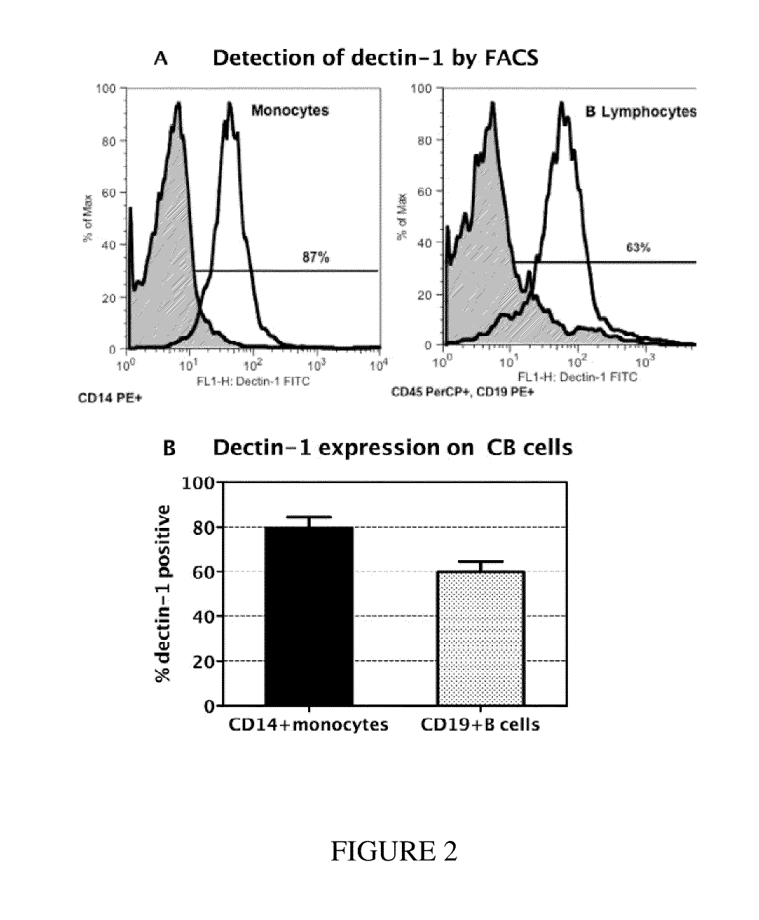

Studies of other beta glucans have shown that monocytes, macrophages, and neutrophils are the principal responding cell type and that this is associated with expression of the dectin-1 receptor on these cells (2, 15, 57, 58, 62). Therefore freshly obtained CB from 12 full term infants were evaluated for expression of dectin-1 by flow cytometry. As shown in FIG. 2, dectin-1 was found to be expressed on both monocytes and B lymphocytes. Panel A shows the flow cytometry of a single representative infant and Panel B shows the group results.

### EXAMPLE 3

Effects of MBG on CB CD34+ cells Homing and Engraftment to NOD/SCID Mouse.

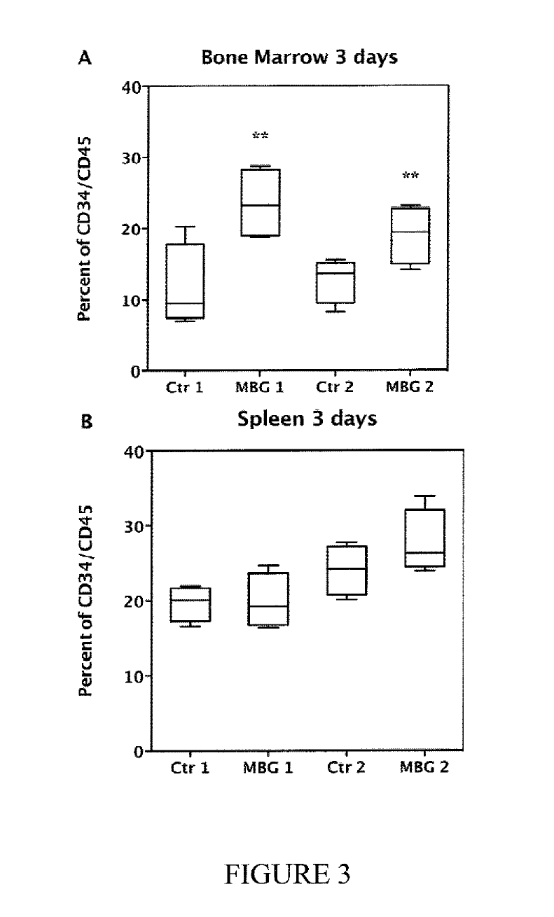

To evaluate the effects of MBG on homing and engraftment, freshly obtained CB enriched for CD34+ precursor cells without expansion were transplated into MBG treated compared to untreated NOD/SCID mice (24, 40). Beta glucan given orally is taken up by intestinal macrophages, which then migrate to the bone marrow where further degradation of the beta glucan occurs (41). The purpose of these experiments was to determine if giving MBG by oral supplementation to the recipient mouse would affect homing and engraftment. Three independent experiments were carried out using 3 to 8 mice in each defined group (n=36 mice); 16 mice were treated with MBG and compared to 16 controls. The other 4 mice were used as irradiation controls without transplantation. For each experiment, one unit CB was transplanted to 8 mice. Mice were then randomly divided such that 4 mice were in both the control and MBG groups. Multiple units of CB samples were used for both homing and engraftment studies. Two-way ANOVA was applied to examine the association between CD34+ cell homing and engraftment and MBG treatment while controlling for different cord blood samples transplanted. For the homing studies, enriched human CD34+ cells prepared from CB samples were labeled with CFSE and transplanted into NOD/SCID mice that had been sublethally irradiated on the previous day. At 3 days the results showed that daily oral administration of MBG led to significantly increased numbers of CFSE-labeled CB CD34+ cells when retrieved from NOD/SCID mouse bone marrow after sacrifice regardless of the cord blood sample used (p-value−0.002) as shown in FIG. 3 panel A. In contrast augmentation of human precursor cell recovery in the spleen compared to conventional transplantation was not observed. As shown in FIG. 3 panel B, although CB CD34+ cells retrieved from MBG treated NOD/SCID mouse spleen (SP) were slightly higher on average than the control group using the second unit cord blood sample, the overall percentage of CD34+ cells recovered remained at the same level as in control mice (p-value=0.30). MBG also did not affect recovery of CD34+ CB stem cells in peripheral blood at 3 days compared to conventional transplantation (data not shown). Therefore the studies indicated that MBG augmented CB CD34+ cells homing to bone marrow but not to spleen in this early stage of transplantation.

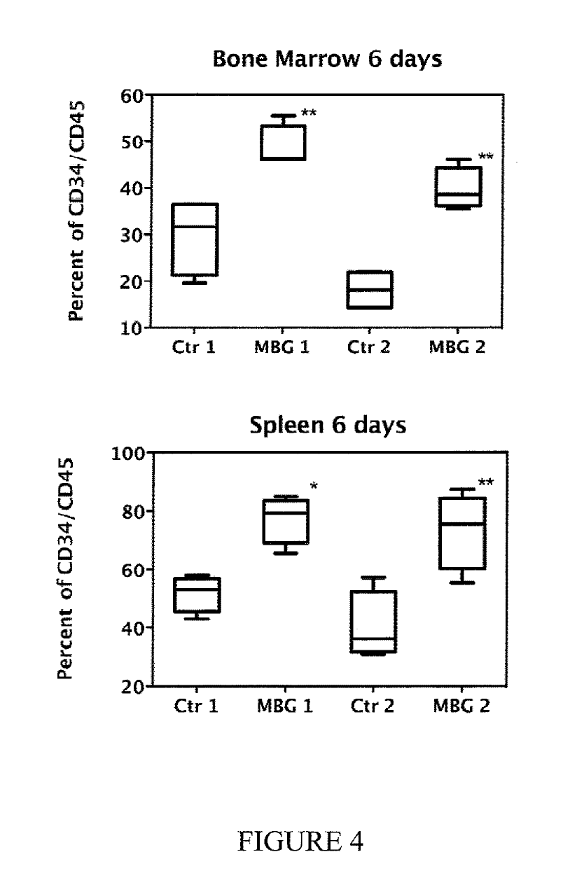

For the engraftment study, at 6 days after transplantation with enriched human CB CD34+ precursor cells, cellular populations were collected from NOD/SCID mice bone marrow and spleen. Cells were prepared as described and analyzed with flow cytometry. Dead cells were excluded using 7-AAD and living CD34+CD45+ cells were selectively gated. As clearly shown in FIG. 4 panel A, after 6 days, the percentages of human cord blood identified by co expression of CD34+CD45+cells retrieved from MBG treated NOD/SCID mice (n=8) bone marrow were very significantly higher than those from control groups (n=8), regardless of the different units of CB used (p-value<0.001). Similarly, as shown in FIG. 4 panel B, the percentages of human CD34+ CD45+ cells retrieved from MBG treated NOD/SCID mouse spleens were very significantly higher than those from the control groups of mice not treated with MBG (p<0.001).

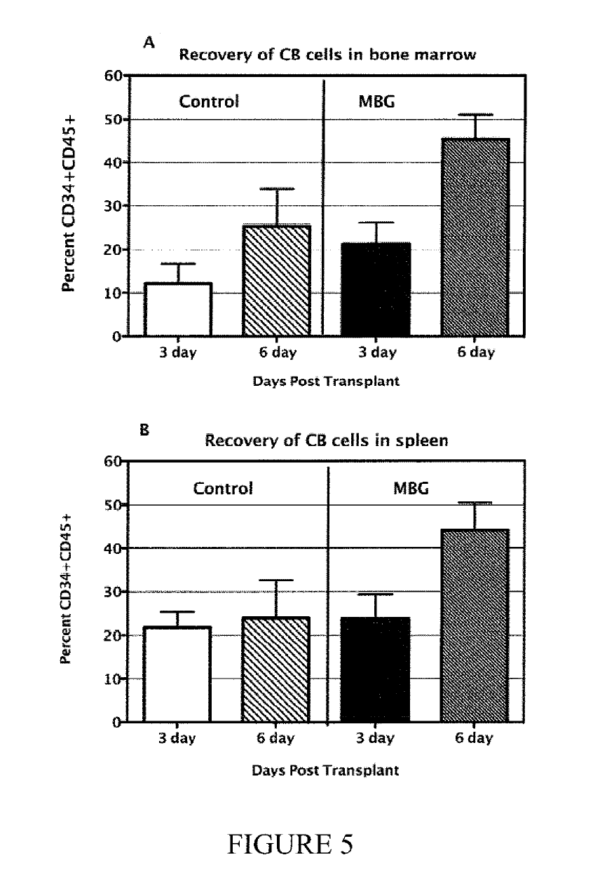

When the effects of transplantation were combined for different cord bloods and compared as groups, the results clearly showed that MBG enhanced homing of human CD34+CD45+ cells to bone marrow compared to conventional transplantation as shown in FIG. 5, panel A. Overall the percentage of human CD34+CD45+ precursor cells increased 1 fold by 6 days in mouse bone marrow and spleen compared to 3 days, for both the control group and the MBG group treated group. In contrast as shown in panel B, there was no effect of MBG on the level of CD34+CD45+ CB cells in spleen at 3 days while at 6 days this population showed a much greater increase in the MBG group compared to the untreated group. This could have reflected an effect of MBG on enhancement of human stem cell proliferation in the spleen.

### EXAMPLE 4

These studies are the first to show that a beta glucan, as described here for MBG, promotes the expansion of human umbilical cord blood CD34+ precursor cells ex vivo and enhances human CD34+ precursor cell homing and engraftment in the NOD/SCID mouse. Compared to conventional expansion media, the dose dependent effect of MBG on expansion of the CD34+ cell population containing myeloid committed HPC progenitor cell was highly significant. The potential relevance for engraftment was evaluated in the xenograft NOD/SCID mouse model assay for human transplantation. Mice were given MBG by oral administration with the intent of influencing the bone marrow microenvironment in the recipient. As shown by recovery of more human cells from mouse bone marrow and spleen of treated mice compared to untreated mice, MBG enhanced human CB CD34+ cell homing and engraftment.

Specifically, MBG showed dose dependent expansion of the CD34+ CD33+ CD38− progenitor cell population, which includes the myeloid committed HPC. The maximum effect was observed at MBG 100 μg/ml. MBG treatment also led to expansion of the uncommitted HSC stem cell identified as CD34+CXCR4+CD38− but these differences were not significant and two way ANOVA analysis suggested an interactive effect.

The mechanism of action in the CD34+ precursor cell expansion studies shown here may involve MBG activation of G-CSF production by CD33+ cord blood monocytes and could require dectin-1. The effects of MBG on HPC are likely to be indirect and mediated by cells that express dectin-1 or other beta glucan receptors such as CR3.

Studies of other beta glucans have shown that monocytes, macrophages, and neutrophils are the principal target cell types and that response is associated with expression of the dectin-1 receptor on these cells. Dectin-1 is a germline-encoded pattern recognition receptor that is analogous to members of the TLR family, and can mediate phagocytosis, production of reactive oxygen intermediates and also interact with TLR signals to induce inflammatory response. Human dectin-1 is widely expressed on myeloid cells, dendritic cells, B cells and a subpopulation of T cells. Data shown in Examples 2-3 confirm that dectin-1 is strongly expressed on both CB monocytes and B cells. Whether dectin-1 is expressed on CD34+ precursor cells or early hematopoietic progenitor cells after expansion is unknown. Since major fungal pathogens such as Candida albicans, Aspergillus niger, Pneumocystis carinii and also Cryptococcus neoforms, Histoplasma capsulatum express beta glucans which directly elicit immune response, botanical beta glucans appear to have potential as natural agonists for the host defense system.

Without being bound to any particular theory, it is possible that MBG supports bone marrow recovery in the NOD/SCID mouse and that recruitment of human cord blood cells to the mouse bone marrow and spleen is part of this process.

### REFERENCES

1. Brown, G D and Gordon, S, Immune recognition. A new receptor for beta-glucans, Nature 413, 36-37, 2001.
2. Brown, G D, Herre, J, Williams, D L, Willment, J A, Marshall, A S and Gordon, S, Dectin-1 mediates the biological effects of beta-glucans, J Exp Med 197, 1119-1124, 2003.
3. Willment, J A, Gordon, S and Brown, G D, Characterization of the human beta-glucan receptor and its alternatively spliced isoforms, J Biol Chem 276, 43818-43823, 2001.

4. Netea, M G, Brown, G D, Kullberg, B J and Gow, N A, An integrated model of the recognition of Candida albicans by the innate immune system, Nat Rev Microbiol 6, 67-78, 2008.

5. Thornton, B P, Vetvicka, V, Pitman, M, Goldman, R C and Ross, G D, Analysis of the sugar specificity and molecular location of the beta-glucan-binding lectin site of complement receptor type 3 (CD11b/CD18), J Immunol 156, 1235-1246, 1996.
6. Cramer, D E, Allendorf, D J, Baran, J T, Hansen, R, Marroquin, J, Li, B, Ratajczak, J, Ratajczak, M Z and Yan, J, Beta-glucan enhances complement-mediated hematopoietic recovery after bone marrow injury, Blood 107, 835-840, 2006.
7. Harada, T, Kawaminami, H, Miura, N N, Adachi, Y, Nakajima, M, Yadomae, T and Ohno, N, Mechanism of enhanced hematopoietic response by soluble beta-glucan SCG in cyclophosphamide-treated mice, Microbiol Immunol 50, 687-700, 2006.
8. Lin, H, She, Y H, Cassileth, B R, Sirotnak, F and Cunningham Rundles, S, Maitake beta-glucan MD-fraction enhances bone marrow colony formation and reduces doxorubicin toxicity in vitro, Int Immunopharmacol 4, 91-99, 2004.
9. Hashimoto, K, Suzuki, I, Ohsawa, M, Oikawa, S and Yadomae, T, Enhancement of hematopoietic response of mice by intraperitoneal administration of a beta-glucan, SSG, obtained from Sclerotinia sclerotiorum, J Pharmacobiodyn 13, 512-517, 1990.
10. Patchen, M L, Vaudrain, T, Correira, H, Martin, T and Reese, D, In vitro and in vivo hematopoietic activities of Betafectin PGG-glucan, Exp Hematol 26, 1247-1254, 1998.
11. Patchen, M L, MacVittie, T J and Weiss, J F, Combined modality radioprotection: the use of glucan and selenium with WR-2721, Int J Radiat Oncol Biol Phys 18, 1069-1075, 1990.
12. Harada, T, Miura, N, Adachi, Y, Nakajima, M, Yadomae, T and Ohn, N, Effect of SCG, 1, 3-beta-D-glucan from Sparassis crispa on the hematopoietic response in cyclophosphamide induced leukopenic mice, Biol Pharm Bull 25, 931-939, 2002.
13. Turnbull, J L, Patchen, M L and Scadden, D T, The polysaccharide, PGG-glucan, enhances human myelopoiesis by direct action independent of and additive to early-acting cytokines, Acta Haematol 102, 66-71, 1999.
14. Patchen, M L, Liang, J, Vaudrain, T, Martin, T, Melican, D, Zhong, S, Stewart, M and Quesenberry, P J, Mobilization of peripheral blood progenitor cells by Betafectin PGG-Glucan alone and in combination with granulocyte colony-stimulating factor, Stem Cells 16, 208-217, 1998
15. Harada, T and Ohno, N, Contribution of dectin-1 and granulocyte macrophage-colony stimulating factor (GM-CSF) to immunomodulating actions of beta-glucan, Int Immunopharmacol 8, 556-566, 2008.
16. Lin, H, Cheung, S W, Nesin, M, Cassileth, B R and Cunningham-Rundles, S, Enhancement of umbilical cord blood cell hematopoiesis by maitake beta-glucan is mediated by granulocyte colony-stimulating factor production, Clin Vaccine Immunol 14, 21-27, 2007.
17. Gan, OI, Murdoch, B, Larochelle, A and Dick, J E, Differential maintenance of primitive human SCID-repopulating cells, clonogenic progenitors, and long-term culture-initiating cells after incubation on human bone marrow stromal cells, Blood 90, 641-650, 1997.
18. Ballen, K K, New trends in umbilical cord blood transplantation, Blood 105, 3786-3792, 2005.
19. Barker, J N and Wagner, J E, Umbilical-cord blood transplantation for the treatment of cancer, Nat Rev Cancer 3, 526-532, 2003.
20. Wagner, J E, Umbilical cord transplantation, Leukemia 12 Suppl 1, S30-32, 1998.
21. Coulombel, L, Identification of hematopoietic stem/progenitor cells: strength and drawbacks of functional assays, Oncogene 23, 7210-7222, 2004.
22. Broxmeyer, H E, Srour, E F, Hangoc, G, Cooper, S, Anderson, S A and Bodine, D M, High-efficiency recovery of functional hematopoietic progenitor and stem cells from human cord blood cryopreserved for 15 years, Proc Natl Acad Sci U S A 100, 645-650, 2003.
23. Rice, A M, Wood, J A, Milross, C G, Collins, C J, McCarthy, N F and Vowels, M R, Conditions that enable human hematopoietic stem cell engraftment in all NOD-SCID mice, Transplantation 69, 927-935, 2000.
24. Lowry, P A, Shultz, L D, Greiner, D L, Hesselton, R M, Kittler, E L, Tiarks, C Y, Rao, S S, Reilly, J, Leif, J H, Ramshaw, H, Stewart, F M and Quesenberry, P J, Improved engraftment of human cord blood stem cells in NOD/LtSz-scid/scid mice after irradiation or multiple-day injections into unirradiated recipients, Biol Blood Marrow Transplant 2, 15-23, 1996.
25. Kollet, O, Petit, I, Kahn, J, Samira, S, Dar, A, Peled, A, Deutsch, V, Gunetti, M, Piacibello, W, Nagler, A and Lapidot, T, Human CD34(+)CXCR4(−) sorted cells harbor intracellular CXCR4, which can be functionally expressed and provide NOD/SCID repopulation, Blood 100, 2778-2786, 2002.
26. Voermans, C, Kooi, M L, Rodenhuis, S, van der Lelie, H, van der Schoot, C E and Gerritsen, W R, In vitro migratory capacity of CD34+ cells is related to hematopoietic recovery after autologous stem cell transplantation, Blood 97, 799-804, 2001.
27. Peled, A, Petit, I, Kollet, O, Magid, M, Ponomaryov, T, Byk, T, Nagler, A, Ben-Hur, H, Many, A, Shultz, L, Lider, O, Alon, R, Zipori, D and Lapidot, T, Dependence of human stem cell engraftment and repopulation of NOD/SCID mice on CXCR4, Science 283, 845-848, 1999.
28. Yao, C L, Feng, Y H, Lin, X Z, Chu, I M, Hsieh, T B and Hwang, S M, Characterization of serum-free ex vivo-expanded hematopoietic stem cells derived from human umbilical cord blood CD133(+) cells, Stem Cells Dev 15, 70-78, 2006.
29. Gluckman, E, Rocha, V and Chevret, S, Results of unrelated umbilical cord blood hematopoietic stem cell transplant, Transfus Clin Biol 8, 146-154, 2001.
30. Hwang, W Y, Samuel, M, Tan, D, Koh, L P, Lim, W and Linn, Y C, A meta-analysis of unrelated donor umbilical cord blood transplantation versus unrelated donor bone marrow transplantation in adult and pediatric patients, Biol Blood Marrow Transplant 13, 444-453, 2007.
31. Tanavde, V M, Malehorn, M T, Lumkul, R, Gao, Z, Wingard, J, Garrett, E S and Civin, C I, Human stem-progenitor cells from neonatal cord blood have greater hematopoietic expansion capacity than those from mobilized adult blood, Exp Hematol 30, 816-823, 2002.
32. Levac, K, Karanu, F and Bhatia, M, Identification of growth factor conditions that reduce ex vivo cord blood progenitor expansion but do not alter human repopulating cell function in vivo, Haematologica 90, 166-172, 2005.
33. Li, K, Chuen, C K, Lee, S M, Law, P, Fok, T F, Ng, PC, Li, C K, Wong, D, Merzouk, A, Salari, H, Gu, G J and Yuen, P M, Small peptide analogue of SDFI alpha supports survival of cord blood CD34+ cells in synergy with other cytokines and enhances their ex vivo expansion and engraftment into nonobese diabetic/severe combined immunodeficient mice, Stem Cells 24, 55-64, 2006.
34. van Hennik, P B, de Koning, A E and Ploemacher, R E, Seeding efficiency of primitive human hematopoietic cells in nonobese diabetic/severe combined immune deficiency mice: implications for stem cell frequency assessment, Blood 94, 3055-3061, 1999.
35. Cohen J. 1988 Statistical Power Analysis for the Behavioral Sciences. 2nd Ed. Mahwah N J: Lawrence Erlbaum Associates.
36. Dorrell, C, Gan, O I, Pereira, D S, Hawley, R G and Dick, J E, Expansion of human cord blood CD34(+)CD38(−) cells in ex vivo culture during retroviral transduction without a corresponding increase in SCID repopulating cell (SRC) frequency: dissociation of SRC phenotype and function, Blood 95, 102-110, 2000.
37. Vanheusden, K, Van Coppemolle, S, De Smedt, M, Plum, J and Vandekerckhove, B, In vitro expanded cells contributing to rapid severe combined immunodeficient repopulation activity are CD34+38-33+90+45RA, Stem Cells 25, 107-114, 2007.
38. Forraz, N, Pettengell, R and McGuckin, C P, Characterization of a lineage-negative stem-progenitor cell population optimized for ex vivo expansion and enriched for LTC-IC, Stem Cells 22, 100-108, 2004.
39. Peled, T, Mandel, J, Goudsmid, R N, Landor, C, Hasson, N, Harati, D, Austin, M, Hasson, A, Fibach, E, Shpall, E J and Nagler, A, Pre-clinical development of cord blood-derived progenitor cell graft expanded ex vivo with cytokines and the polyamine copper chelator tetraethylenepentamine, Cytotherapy 6, 344-355, 2004.
40. Kerre, T C, De Smet, G, De Smedt, M, Offner, F, De Bosseher, J, Plum, J and Vandekerckhove, B, Both CD34+38+ and CD34+38− cells home specifically to the bone marrow of NOD/LtSZ scid/scid mice but show different kinetics in expansion, J Immunol 167, 3692-3698, 2001.
41. Hong, F, Yan, J, Baran, J T, Allendorf, D J, Hansen, R D, Ostroff, G R, Xing, P X, Cheung, N K and Ross, G D, Mechanism by which orally administered beta-1,3-glucans enhance the tumoricidal activity of antitumor monoclonal antibodies in murine tumor models, J Immunol 173, 797-806, 2004.
42. Brunstein, C G, Setubal, D C and Wagner, J E, Expanding the role of umbilical cord blood transplantation, Br J Haematol 137, 20-35, 2007.
43. Wagner, J E, Barker, J N, DeFor, T E, Baker, K S, Blazar, B R, Eide, C, Goldman, A, Kersey, J, Krivit, W, MacMillan, M L, Orchard, P J, Peters, C, Weisdorf, D J, Ramsay, N K and Davies, S M, Transplantation of unrelated donor umbilical cord blood in 102 patients with malignant and nonmalignant diseases: influence of CD34 cell dose and HLA disparity on treatment-related mortality and survival, Blood 100, 1611-1618, 2002.
44. Ueda, T, Yoshida, M, Yoshino, H, Kobayashi, K, Kawahata, M, Ebihara, Y, Ito, M, Asano, S, Nakahata, T and Tsuji, K, Hematopoietic capability of CD34+ cord blood cells: a comparison with CD34+ adult bone marrow cells, Int J Hematol 73, 457-462, 2001.
45. Shpall, E J, Quinones, R, Giller, R, Zeng, C, Baron, A E, Jones, R B, Bearman, S I, Nieto, Y, Freed, B, Madinger, N, Hogan, C J, Slat-Vasquez, V, Russell, P, Blunk, B, Schissel, D, Hild, E, Malcolm, J, Ward, W and McNiece, I K, Transplantation of ex vivo expanded cord blood, Biol Blood Marrow Transplant 8, 368-376, 2002.
46. Kim, S K, Koh, S K, Song, S U, Shin, S H, Choi, G S, Kim, W C, Lee, M H, Seoh, J Y, Park, S K and Fraser, J K, Ex vivo expansion and clonality of CD34+ selected cells from bone marrow and cord blood in a serum-free media, Mol Cells 14, 367-373, 2002.
47. Mazurier, F, Doedens, M, Gan, O I and Dick, J E, Rapid myeloerythroid repopulation after intrafemoral transplantation of NOD-SCID mice reveals a new class of human stem cells, Nat Med 9, 959-963, 2003.
48. McKenzie, J L, Gan, O I, Doedens, M and Dick, J E, Human short-term repopulating stem cells are efficiently detected following intrafemoral transplantation into NOD/SCID recipients depleted of CD122+ cells, Blood 106, 1259-1261, 2005.
49. Blom, B and Spits, H, Development of human lymphoid cells, Annu Rev Immunol 24, 287-320, 2006.
50. Zhao, P, Liu, W and Cui, Y, Rapid immune reconstitution and dendritic cell engraftment post-hone marrow transplantation with heterogeneous progenitors and GM-CSF treatment, Exp Hematol 34, 951-964, 2006.
51. Hogan, C J, Shpall, E J and Keller, G, Differential long-term and multilineage engraftment potential from subfractions of human CD34+ cord blood cells transplanted into NOD/SCID mice, Proc Natl Acad Sci U S A 99, 413-418, 2002.
52. Cramer, D E, Wagner, S, Li, B, Liu, J, Hansen, R, Reca, R, Wu, W, Surma, E Z, Laber, D A, Ratajczak, M Z and Yan, J, Mobilization of hematopoietic progenitor cells by yeast-derived beta-glucan requires activation of matrix metalloproteinase-9, Stem Cells 26, 1231-1240, 2008.
53. Cheung, N K and Modak, S, Oral (1-->3),(1-->4)-beta-D-glucan synergizes with antiganglioside GD2 monoclonal antibody 3F8 in the therapy of neuroblastoma, Clin Cancer Res 8, 1217-1223, 2002.
54. Patchen, M L, MacVittie, T J, Solberg, B D and Souza, L M, Survival enhancement and hemopoietic regeneration following radiation exposure: therapeutic approach using glucan and granulocyte colony-stimulating factor, Exp Hematol 18, 1042-1048, 1990.
55. Vos, A P, M'Rabet, L, Stahl, B, Boehm, G and Garssen, J, Immune-modulatory effects and potential working mechanisms of orally applied nondigestible carbohydrates, Crit Rev Immunol 27, 97-140, 2007.
56. Vetvicka, V, Dvorak, B, Vetvickova, J, Richter, J, Krizan, J, Sima, P and Yvin, J C, Orally administered marine (1-->3)-beta-D-glucan Phycarine stimulates both humoral and cellular immunity, Int J Biol Macromol 40, 291-298, 2007.
57. Taylor, P R, Brown, G D, Reid, D M, Willment, J A, Martinez-Pomares, L, Gordon, S and Wong, S Y, The beta-glucan receptor, dectin-1, is predominantly expressed on the surface of cells of the monocyte/macrophage and neutrophil lineages, J Immunol 169, 3876-3882, 2002.
58. Willment, J A, Marshall, A S, Reid, D M, Williams, D L, Wong, S Y, Gordon, S and Brown, G D, The human beta-glucan receptor is widely expressed and functionally equivalent to murine Dectin-1 on primary cells, Eur J Immunol, 2005.
59. Borchers, A T, Keen, C L and Gershwin, M E, Mushrooms, tumors, and immunity: an update, Exp Biol Med (Maywood) 229, 393-406, 2004.
60. Week, M M, Appel, S, Werth, D, Sinzger, C, Bringmann, A, Grunebach, F and Brossart, P, hDectin-1 is involved in uptake and cross-presentation of cellular antigens, Blood 111, 4264-4272, 2008.
61. Gow, N A, Netea, M G, Munro, C A, Ferwerda, G, Bates, S, Mora-Montes, H M, Walker, L, Jansen, T, Jacobs, L, Tsoni, V, Brown, G D, Odds, F C, Van der Meer, J W, Brown, A J and Kullberg, B J, Immune Recognition of Candida albicans beta-glucan by Dectin-1, J Infect Dis 196, 1565-1571, 2007.
62. Dennehy, K M and Brown, G D, The role of the {beta}-glucan receptor Dectin-1 in control of fungal infection, J Leukoc Biol 82, 253-258, 2007.
63. Ishibashi, K, Yoshida, M, Nakabayashi, I, Shinohara, H, Miura, N N, Adachi, Y and Ohno, N, Role of anti-beta-glucan antibody in host defense against fungi, FEMS Immunol Med Microbiol 44, 99-109, 2005.
64. Krajicek, B J, Limper, A H and Thomas, C F, Jr., Advances in the biology, pathogenesis and identification of Pneumocystis pneumonia, Curr Opin Pulm Med 14, 228-234, 2008.
65. Akamatsu, N, Sugawara, Y, Kaneko, J, Tamura, S and Makuuchi, M, Preemptive treatment of fungal infection based on plasma (1-->3) beta-D-glucan levels after liver transplantation, Infection 35, 346-351, 2007.
66. Rappleye, C A, Eissenberg, L G and Goldman, W E, Histoplasma capsulatum alpha-(1,3)-glucan blocks innate immune recognition by the beta-glucan receptor, Proc Natl Acad Sci U S A 104, 1366-1370, 2007.
67. Acosta-Rodriguez, E V, Rivino, L, Geginat, J, Jarrossay, D, Gattorno, M, Lanzavecchia, A, Sallusto, F and Napolitani, G, Surface phenotype and antigenic specificity of human interleukin 17-producing T helper memory cells, Nat Immunol 8, 639-646, 2007.
68. Schwarzenberger, P, Huang, W, Oliver, P, Byrne, P, La Russa, V, Zhang, Z and Kolls, J K, 11-17 mobilizes peripheral blood stem cells with short- and long-term repopulating ability in mice, J Immunol 167, 2081-2086, 2001.
69. Tan, W, Huang, W, Gu, X, Zhong, Q, Liu, B and Schwarzenberger, P, IL-17F/IL-17R interaction stimulates granulopoiesis in mice, Exp Hematol, 2008.
70. Lapidot, T, Mechanism of human stem cell migration and repopulation of

NOD/SCID and B2mnull NOD/SCID mice. The role of SDF-1/CXCR4 interactions, Ann N Y Acad Sci 938, 83-95, 2001.

### EXAMPLE 5

In this study, paclitaxel (Ptx) was used to induce neutropenia in B6D2F1mice to test the hypothesis that MBG would hasten neutrophil recovery from chemotoxicity. After a cumulative dose of Ptx (90-120 mg/kg) given to B6D2F1 mice, daily oral MBG (4 or 6 mg/kg), intravenous G-CSF (80 ug/kg) or Ptx alone were compared for effects on the dynamics of leukocyte recovery in blood, CFU-GM activity in bone marrow and spleen, and granulocyte/monocyte production of reactive oxygen species (ROS). Leukocyte counts declined less in Ptx+MBG mice compared to Ptx-alone (p=0.024) or Ptx+G-CSF treatment (p=0.031). Lymphocyte levels were higher after Ptx+MBG but not Ptx+G-CSF treatment compared to Ptx alone (p<0.01). MBG increased CFU-GM activity in bone marrow and spleen (p<0.001, p=0.002) 2 days after Ptx. After two additional days (Ptx post day 4), MBG restored granulocyte/monocyte ROS response to normal levels compared to Ptx-alone and increased ROS response compared to Ptx-alone or Ptx+G-CSF (p<0.01, both). The studies indicate that oral MBG promoted maturation of HPC to become functionally active myeloid cells and enhanced peripheral blood leukocyte recovery after chemotoxic bone marrow injury. The conclusion is that MBG is beneficial as an adjunct for the reduction of the hematologic toxicity of chemotherapy associated with cancer treatment.

The experiments described in the following Examples were conducted to determine if a beta-glucan extract from the G. frondosa mushroom that is orally active and was safely administered to breast cancer patients without inducing changes in peripheral blood counts would stimulate hematopoiesis and enhance recovery from paclitaxel in a mouse model of dose-intensive chemotherapy [84].

### EXAMPLE 6

### Materials and Methods

Chemicals and Reagents: Maitake mushroom beta-glucan (MBG), also known as D fraction and characterized by a 1, 6 main chain with 1, 3 branches, is an extract from fruit body of Maitake mushroom (Grifola frondosa), which was made according to the methods described in U.S. Pat. No. 5,854,404 (corresponding to Japan Patent No. 2859843), and was provided by Yuikiguni Maitake Corp. through the Tradeworks group. MBG was stored at 4° C. in the dark until use. The lot of MBG used here was tested by NAMSA for endotoxin by limulus amebocyte lysate (LAL) assay and none was found (limit of detection−0.012 EU/mg, equivalent to medium). MBG was dissolved in RPMI 1640 with 25 mM HEPES buffer and prepared at a concentration of 20 mg/ml, sterilized by filtration through 0.2 um cellulose acetate low protein binding membrane and stored at −20° C. The stock solution was diluted in RPMI 1640 for use. Pharmaceutical grade Paclitaxel (Ptx) was obtained from Mayne Pharma, USA. Neupogen (G-CSF) was from Amgen (Thousand Oaks, Calif.).

MBG characterization, composition, and purity: The fruit bodies of dried G. frondosa were extracted with distilled water at 121° C., and the resulting aqueous extraction was precipitated by adding ethanol for a final concentration of 45% (v/v), and after standing at 4 ° C. for 12 hours, the precipitates were removed by filtration. Additional ethanol was added to the filtrate for a final concentration of at least 80% (v/v), the solution was allowed to stand at 4 ° C. and the resulting precipitate (MBG) was dark brown to black in color. MBG is a glucan/protein complex deduced by the positive response in anthrone reaction and ninhydrin reaction, and the glucan/protein ratio was 96:4. The molecular weight is distributed around 1,000,000z as determined by gel filtration chromatography on a TSK gel GMPW_{XL} column. The protein moiety was characterized by an automatic amino acid analyzer, as consisting of glutamic acid, aspartic acid, alanine, leucine, lysine, glycine, isoleucine, serine, valine, proline, threonine, arginine, phenylalanine, tyrosine, histidine, methionine, and cysteine.

Glycosyl composition analysis was performed by combined gas chromatography/mass spectrometry (GC/MS) of the per-O-trimethylsilyl(TMS) derivatives of the monosaccharide methyl glycosides produced from the sample by acidic methanolysis. The experiment was performed on an HP 5890 GC interfaced to a 5970 MSD, using a Supelco DB5 fused silica capillary column. Methyl glycosides were prepared by methanolysis in 1 M HCl in methanol at 80° C. (18-22 hours), followed by re-N-acetylation with pyridine and acetic anhydride in methanol. The samples were then per-O-trimethylsilylated by treatment with Tri-Sil (Pierce) at 80° C. (0.5 hours). These procedures were essentially the same as previously described [97, 98]. The glycosyl composition consists of glucose, galactose, and mannose, at the ratio of 96.2, 1.5 and 2.3%, respectively, expressed as mole percent of total carbohydrate. Trace amount of ribose was also detected.

Mice: B6D2F1 mice female, 6-8 weeks old were purchased from Jackson Laboratory, and were maintained in a pathogen-free facility at Memorial Sloan-Kettering Cancer Center (MSKCC, New York, N.Y.) with access to fresh water and food ad libitum. Certified Rodent Diet #5053 (Lab Diet) was used. All animal experiments were approved by the MSKCC Animal Care Committee.
Treatments: Ptx was intraperitoneally injected (i.p) at 30 mg/kg/day over 3-7 days with cumulative dose of 90-120 mg/kg to mice. Neupogen (G-CSF) was given i.v. at 80 microg/kg at 24 hrs after the last dose of Ptx. MBG was administered orally at 4 mg/kg/day or 6 mg/kg/day (about 100-150 microg/mouse) by gavage on every experimental day in the Ptx+MBG groups. Mice were weighed each day. Blood was taken from the tail vein for complete blood cell (CBC) counts. Sacrifice was performed by the CO₂ method.

Complete Blood Cell Count: Approximately 20 microL of blood was taken from the tail vein after warming, for assessment of CBC by automated differential analysis using the Hemavet instrument after instrument standardization.

Bone Marrow: Mouse bone marrow cells were collected as previously described [94]. Briefly, mouse bone marrow cells were collected from femoral shafts by flushing with 3 mL of cold RPMI-1640. The cell suspensions were passed up and down six times through an 18-gauge needle in RPMI-1640 to disperse clumps. Adherent bone marrow cells were removed after incubation at 37° C., 5% CO₂ for 24 hrs in RPMI-1640 containing 20% FBS, and non-adherent cells were collected and used as described.

Spleen: Mouse SP cells were collected with smearing between two sterile glass-slides a few times in˜3 mL RPMI-1640 medium. Cell clumps were dispersed as described above.

Colony Forming Unit (CFU-GM) Assays: The colony-forming assay was carried out under defined conditions (StemCell Technologies Inc. Vancouver, Canada) as described previously [94]. Briefly, bone marrow cells were placed in premixed methylcellulose culture medium (Methocult M3234, StemCell Technologies Inc.; Vancouver, Canada). Final adjusted concentrations were 1% methylcellulose, 15% FBS, 1% BSA, 10 microg/ml insulin, 200 microg/ml transferrin, 10⁻⁴ M 2-mercaptoethanol and 2 mM L-glutamine. Recombinant murine IL-3 (Intergen Company, Purchase, N.Y.), and recombinant human G-CSF (Neupogen, AMGEN, Thousand Oaks, Calif.) were added at 10 ng/ml and 500 ng/ml. The bone marrow cell (5×10⁵ cells/ml, 0.3 ml), or spleen cell (2×10⁶ cells/ml, 0.3 ml) suspensions were added to complete mixed culture medium (2.7 ml), vortexed, and plated in Petri dishes (Falcon, Becton Dickinson), 1.1 ml/dish. Then all cultures were incubated in a water-saturated, 37° C., 5% CO2 atmosphere for 7 days. CFU-GM colonies of 50 or more cells were scored by inverted microscope.

Flow Cytometric Oxidative Burst Assay: Peripheral blood was obtained by retro orbital bleeding; about 400 microL blood were collected into heparinized tubes. The respiratory burst assay was performed as described [99] with modifications. Briefly, 100 microL blood aliquots were added to each tube, wash buffer or stimuli (opsonized E. coli, fMLP (N-formylmethionyl-leucyl-phenylalanine) were added at 20 microL. Tubes were incubated in a water bath at 37° C. for 10 mins, then 20 microL of dihydrorhodamine (DHR-123) was added and tubes were vortexed, and then incubated for another 10 mins. Red blood cells were lysed and removed after washing once. R-PE conjugated rat anti-mouse Ly-6G and Ly-6C (Gr-1, BD Biosciences) antibody was added in the presence of Mouse BD Fc Block (purified anti-mouse CD16/CD32 monoclonal antibody, 2.4G2, BD Biosciences) at 1 μg/million cells, and incubated at room temperature in the dark for 15 mins. Cells were washed; supernatants removed and 200 μL of DNA staining solution was added. The samples were analyzed by flow cytometer (FACSCalibur) using CellQuest. The initial gate was set with Gr-1 to identify granulocytes/monocytes; the percentage of responding cells was then analyzed with FlowJo software.

Statistical Analysis: Data are presented as mean percentage ±SD or mean counts ±SD. To identify the appropriate dose of Ptx, ANCOVA was used to examine significance of variation in average cell counts or percentage of change from baseline across all days for the cell type of interest in a treatment group and possible within-mouse correlation was adjusted by including mouse ID as a covariate. If overall significance by ANCOVA was shown, Tukey's method was used to determine between-day significance. To assess significance of variation across dose groups (including untreated) over the treatment interval, two-way ANOVA was used and then Bonferroni method was used to adjust for between-group multiple comparisons. In the studies of blood cell recovery, the decrease in cell count was calculated as compared to the corresponding baseline during the treatment for each mouse. The averages of the group decreases across different treatment groups were compared using two-way ANOVA to determine significance. Two sample t-tests were used to compare the difference in average percentage change at a given time point between any two treatment groups. Tukey's or Bonferroni method was used to adjust p-values for multiple comparisons where appropriate. The analyses were carried out using GraphPad Prism software version 5.01. Additional information is provided in the text.

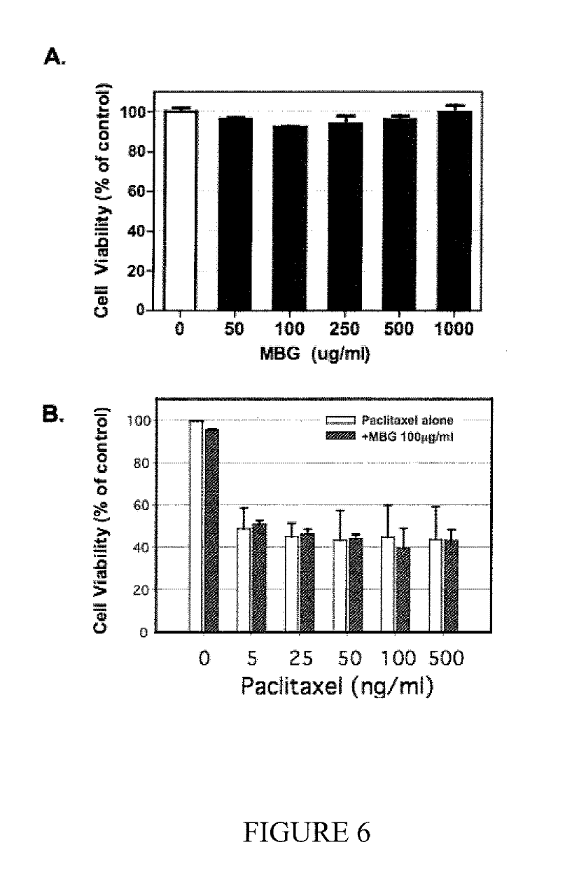

Effect of MBG on Growth of MCF-7 Tumor Cell Line and Paclitaxel Cytotoxicity: Before undertaking studies to evaluate the effect of MBG on response to paclitaxel, the effect of MBG on the growth of the breast cancer tumor cell line MCF-7 and during treatment with paclitaxel was evaluated. It was determined that MBG did not stimulate or inhibit the growth of tumor cell lines or affect the response to chemotherapeutic drugs [95]. FIG. 6 shows that MBG did not affect the viability of MCF-7 or inhibit the cytotoxic effect of paclitaxel.

### EXAMPLE 7

Paclitaxel-Induced Leukopenic Mouse Model

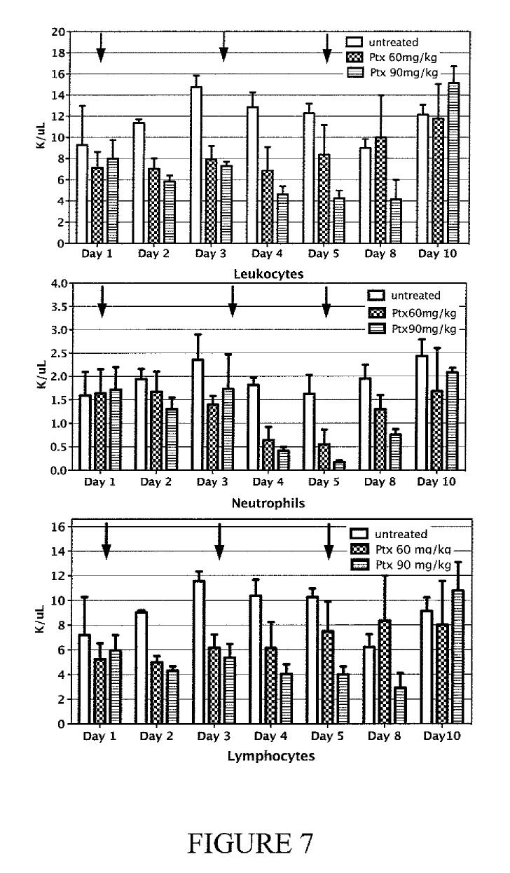

To determine the dose of Ptx required for induction of acute hematotoxicity that also permitted spontaneous recovery, fractionated dosing at 10 to 30 mg/kg was given 3 times every other day to groups of mice. After doses greater than 15 mg/kg were found effective, we compared cumulative doses of 60 and 90 mg/kg to untreated mice (n=4 each group). Serial CBCs were obtained from each mouse. Mean absolute numbers of leukocytes, neutrophils, and lymphocytes are shown in FIG. 7. Variation was evaluated by one-way ANCOVA and within mouse correlation was included as the covariate. Pair-wise differences were evaluated by Tukey's posttest. Treatment group variation was assessed by two-way ANOVA over the interval followed by between-group comparisons using Bonferroni posttests. As shown in FIG. 7, leukocyte, neutrophil, and lymphocyte numbers did not decline in the untreated group.

Leukocytes: A cumulative Ptx dose of 90 mg/kg, but not 60 mg/kg, produced significant changes in leukocyte numbers (one-way ANCOVA; p<0.0001). Compared to baseline, leukocyte numbers after 90 mg/kg Ptx were lower on day 4 (p<0.05), day 5 (p<0.01) and 3 days after Ptx was stopped on day 8 (p<0.05) by Tukey's posttests. Differences across treatment groups were significant (two-way ANOVA, p<0.0001). The 60 mg/kg Ptx group had fewer leukocytes on days 3, 4 (p<0.0001) or day 5 (p<0.05) and the 90 mg/kg Ptx group had lower leukocyte numbers on days 3, 4, and 5 (p<0.0001), compared to untreated mice (Bonferroni). Rebound in leukocytes occurred later after the higher Ptx dose compared to the lower dose.

Neutrophils: After Ptx at 60 mg/kg the overall drop in neutrophil counts across all days was significant (p<0.01, ANCOVA) but no pair-wise comparisons were significant. In contrast at 90 mg/kg, Ptx caused a greater overall change in neutrophil numbers (p<0.0001, ANCOVA) and counts were significantly lower on days 4 and 5 compared to baseline (p<0.001, p<0.0001, Tukey's). Significant variation among treatment groups was observed (p<0.0001, two-way ANOVA). Compared to the untreated group, neutrophils were more reduced after the higher dose of Ptx (p<0.001) and the counts were still down on day 8 (p<0.01).

Lymphocytes: Lymphocyte numbers varied significantly across time only after 90 mg/kg Ptx (p<0.001, ANCOVA). Lymphocytes were lower on day 8 compared to baseline (p<0.05, Tukey's). Overall between-group variation was also significant (p<0.0001, two-way ANOVA). Lymphocyte numbers were lower over days 3, 4, and 5 (p<0.001) after 90 mg/kg compared to the untreated group and on day 8 compared to 60 mg/kg Ptx (p<0.001, Bonferroni).

### EXAMPLE 8

Effect of MBG on Recovery of Hematopoietic Progenitor CFU-GM Activity after Ptx

Recovery of peripheral blood counts after chemotherapy depends upon emergency bone marrow hematopoiesis after chemotoxic injury. To determine if MBG would enhance CFU-GM activity in bone marrow and spleen after Ptx treatment, mice (n=4 each group) received 4 doses of Ptx at 30 mg/kg/day every other day for a cumulative dose of 120 mg/kg. Another group received the same Ptx dose and was also given daily oral MBG at 4 mg/kg/day, starting on the first day of Ptx and throughout the experimental period. The choice of initial dose level was based on a previous study (described hereinabove) showing that 4 mg/kg was sufficient to enhance homing and engraftment of human CD34+ cells in a xenograft model. Bone marrow and spleen cells were collected for CFU-GM colony forming assays performed ex vivo two days after the last Ptx injection. For each sample, colony counts were obtained from cultures plated in quadruplicate. Measurements from the same mouse were considered to be in the same cluster, and repeated measures ANOVA was used to compare the CFU-GM numbers in each tissue after log transformation was applied.

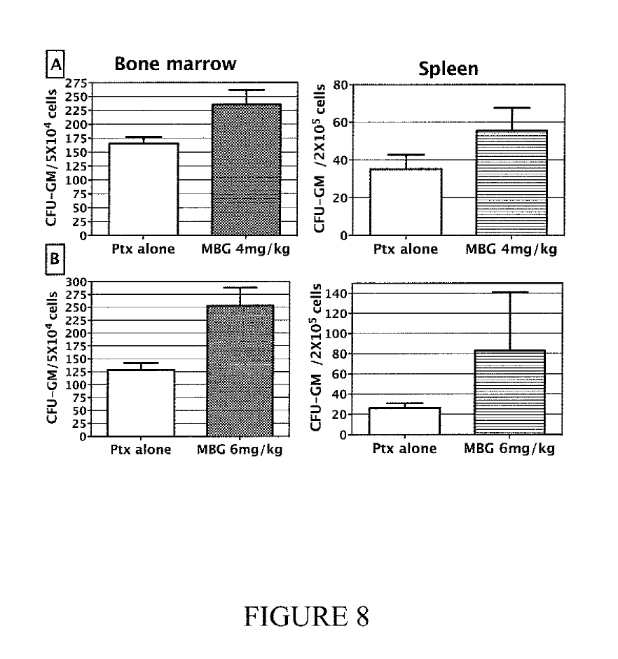

FIG. 8, panel A shows that Ptx+MBG treatment led to higher CFU-GM activity in both bone marrow and spleen compared to Ptx alone (p<0.001, p=0.002, respectively). The dose of MBG was then increased to 6 mg/kg/day with the same Ptx regimen. As shown in FIG. 8, panel B, bone marrow from Ptx+MBG treated mice had greater CFU-GM activity compared to Ptx alone (p=0.003). Compared to Ptx-alone, Ptx+MBG at 6 mg/kg also increased CFU-GM activity in the spleen, but differences were not statistically significant (p=0.07) due to greater variation within the group and smaller sample size.

### EXAMPLE 9

Effect of MBG and G-CSF on Dynamics of Leukocyte Recovery after Ptx Treatment:

Since MBG enhanced bone marrow and spleen CFU-GM activity after Ptx, the relationship to the dynamics of peripheral blood leukocyte recovery was investigated. Treatment with Ptx+MBG or Ptx plus G-CSF was compared to Ptx-alone in 3 groups (n=6, each group). 30 mg/kg/day Ptx was given over 3 consecutive days (90 mg/kg cumulative dose) to each group. Mice given Ptx+MBG received 6 mg/kg/day orally each day from day 1 of chemotherapy and daily thereafter. The Ptx+G-CSF group received one dose of intravenous G-CSF (80 ug/kg), 24 hrs after the last Ptx dose. Changes in absolute numbers of leukocytes (white blood cell count) from baseline were evaluated from post-day 1 to post-day 8 after chemotherapy. For each mouse, percent change in counts from baseline was calculated. Change from baseline across days within a treatment group was assessed by ANCOVA and within-mouse correlation was included as appropriate. Paired t-tests were used to compare any two time-points. Differences among treatment groups across the experimental period were assessed by two-way

ANOVA followed by two sample t-tests for pair-wise comparisons. Tukey's or Bonferroni methods were used as appropriate.

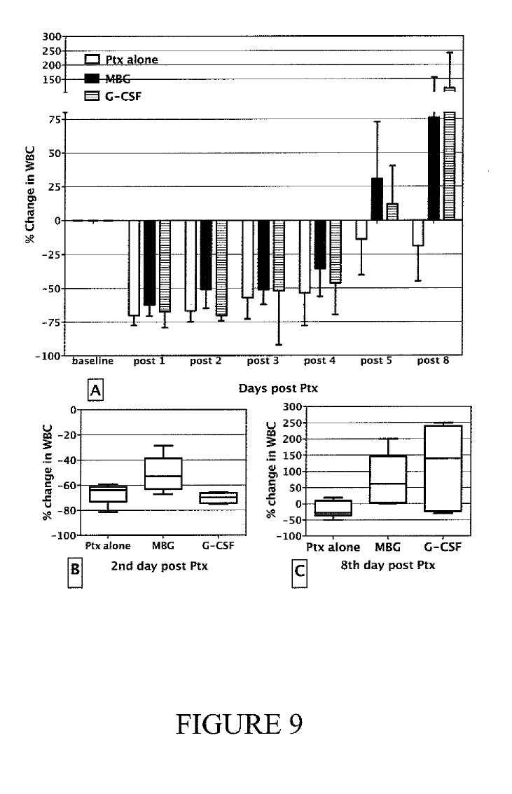

Leukocytes: Treatment with 90 mg/kg Ptx-alone caused a marked reduction in leukocyte numbers. Overall changes from baseline were significant (p<0.0001 by ANCOVA). As shown in FIG. 9, panel A, numbers declined by 70% on post-day 1. Differences compared to baseline were significant for post-days 1-4 (p<0.0001). Counts improved by post-day 5 and 8 compared to post-day 1 (p<0.001) but were not restored to baseline levels. Leukocyte numbers were also reduced in both Ptx+MBG and Ptx+G-CSF groups. Change from baseline was significant across time for both (p<0.0001). Leukocytes were lower over the first 4 post-Ptx days for both groups but recovery began earlier compared to Ptx-alone. For the Ptx+MBG group, leukocytes were reduced on post-days 1-3 (p<0.01), or 4 (p=0.05) compared to baseline. By post-day 5, levels increased compared to post-days 1-3 (p<0.005), or 4 (p<0.03). For the Ptx+G-CSF group, leukocytes were reduced through post-day 4 with average counts below baseline. Compared to baseline, counts were lower on post-day 1 and 2 (p<0.0001), 3 (p<0.001), and 4, (p<0.05). After Ptx+G-CSF, leukocyte numbers increased by post-day 5 compared to post-day 1 and 2. By post day 8, the average count was significantly higher compared to day 1, 2, 3 (p<0.0001), 4 (p<0.001) or 5 (p=0.04). Comparison among the treatment groups showed that the maximum decrease (nadir) in leukocytes was least in the Ptx+MBG group. Differences were significant compared to Ptx-alone (p=0.024) and to Ptx+G-CSF (p=0.031). As shown in FIG. 9 panel B, on post-day 2 the effect of G-CSF was not observed, while the ameliorating effect of MBG on Ptx treatment was evident. The decline in leukocytes after Ptx+MBG was less than after Ptx-alone (p<0.05) or Ptx+G-CSF (p<0.01). As shown in FIG. 9, panel C, by the 8^{th} day after Ptx treatment, mice in both the Ptx+MBG group and the Ptx+G-CSF group had higher leukocyte counts compared to Ptx-alone (p<0.001).

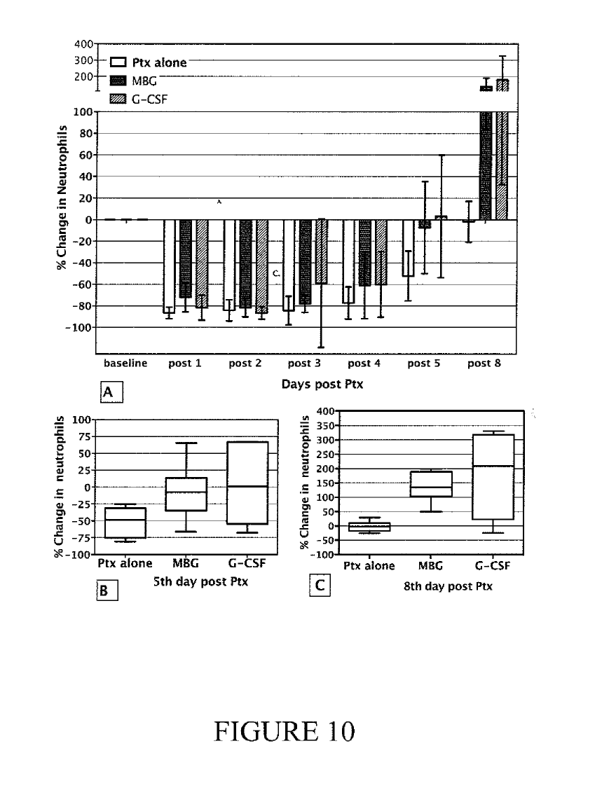

Neutrophils: Neutrophil numbers declined sharply from baseline following Ptx treatment alone. The overall change from baseline was significant (p<0.0001, ANCOVA) as shown in FIG. 10, panel A. The mean decline from baseline was 87% at post-day 1 (p<0.0001, Tukey's). By post-day 5, recovery had begun and neutrophil levels were higher compared to post-days 1, 2, and 3 (p<0.05). Baseline levels were achieved by post-day 8. Neutrophil counts declined in the Ptx+MBG group. The change from baseline was significant (p<0.0001 by ANCOVA) and neutrophil levels were reduced compared to baseline on post treatment days 1-4 (p<0.05, Tukey's). By post-day 5 average neutrophil counts had recovered to baseline level and were higher compared to post-day 1-3 (p<0.05) or 4 (p=0.09). In the Ptx+G-CSF group, neutrophil numbers showed significant overall variation (p<0.0001, ANCOVA). By post-day 5, the neutrophil level was greater than on post-day 1 or 2 (p<0.05). Comparison of average neutrophil counts across treatment groups showed significant variation across the 8-day period (p<0.0001, two- way ANOVA). The treatment groups were also compared over the period of early recovery from chemotherapy (baseline to day 5). Average neutrophil counts varied significantly (p<0.0001, two-way ANOVA). The decrease in neutrophil level in the early recovery period was greater in the Ptx-alone group compared to either Ptx+G-CSF (p<0.01) or Ptx+MBG (p<0.05) treated groups on post-day 5. See FIG. 10 panel B. On the 8^{th} post Ptx day, as shown in FIG. 10 panel C, the neutrophil counts in the Ptx+MBG group were higher compared to Ptx-alone (p<0.04), and comparable to Ptx+G-CSF treatment.

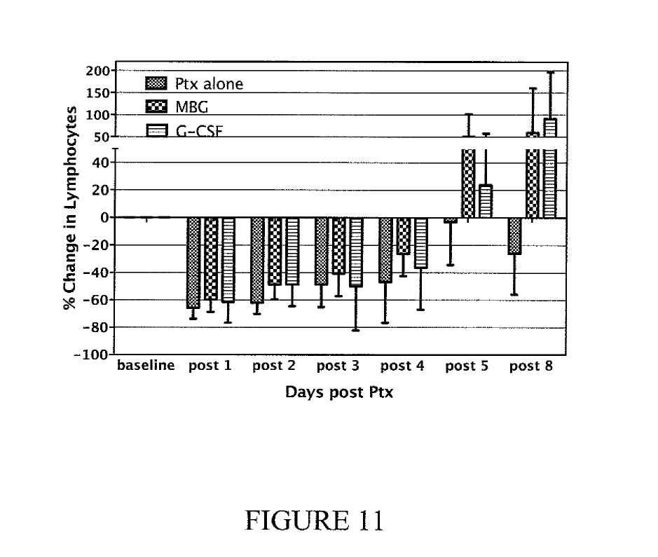

Lymphocytes: Lymphocytes were reduced by more than 50% in all treatment groups after chemotherapy. Overall change from baseline was significant in the Ptx-alone group (p=0.001, ANCOVA). Decline from baseline was significant on post-days 1, 2 (p<0.001, Tukey's) and 3, 4 (p<0.01). While neutrophils were increased on post-day 5 compared to post-day 1 or 2 (p<0.01), baseline levels were not achieved, even by post-day 8 in the Ptx-alone group. See FIG. 11. Lymphocytes declined on post-day 1, 2, or 3 when compared to baseline in the Ptx+MBG group (p<0.001, p<0.02, respectively, Tukey's). By post-day 4 lymphocytes were not statistically different from baseline. By post-day 5, levels were higher than on post-days 1-4 (p<0.0001, Tukey's). After Ptx+G-CSF, lymphocyte recovery began on post-day 4, and by post-day 5 these levels were higher compared to post-days 1-3 (p<0.05, Tukey's) or 4 (p=0.06). Overall differences in lymphocyte numbers across treatment groups were also significant (p<0.01, two-way ANOVA). Lymphocyte levels were higher in the Ptx+MBG group but not the Ptx+G-CSF treated group compared to Ptx alone on post day 5 (p<0.01, Bonferroni).

### EXAMPLE 10

Effects of Ptx, MBG and G-CSF on Monocytes, Erythrocytes, and Platelets

The relative effects of MBG and G-CSF compared to Ptx-alone on peripheral blood monocytes, erythrocytes, hemoglobin and platelets counts were also examined, and differences were found.

Monocytes: Monocyte numbers declined in all treatment groups but were restored by post-day 8. Overall percent change from baseline across the experimental period was significant for all (p<0.0001, ANCOVA). For both Ptx-alone and Ptx+MBG groups, the lowest point occurred on post-day 3 when monocyte counts were 77.6±13% below baseline for Ptx-alone and 84.9±7.3% below baseline for the Ptx+MBG group. For the Ptx+G-CSF group, counts were lowest on post-day 2 at 90.5±4.0% below baseline. Variation among treatment groups across was significant (p<0.0001, two way ANOVA). Monocyte levels were more suppressed in the Ptx+G-CSF group compared to the Ptx-alone group on post-day 4 (p<0.001 and post-day 5 (p<0.01, Bonferroni).

Erythrocytes and Hemoglobin: For all groups, the maximum drop in mean absolute RBC counts occurred on post-day 5 and levels did recover to baseline. Variation in average absolute erythrocyte number was significant for each treatment group (p<0.0001). The baseline mean absolute RBC count in the Ptx-alone group was 10.4±0.3 K/microL, dropped to 5.1±1.7 K/microL at post-day 5 (p<0.0001), and rose to 7.9±0.9 K/microL by post-day 8. The baseline RBC count in the Ptx+MBG group was 11.1±0.4 K/microL, and fell to 5.6±1.0 K/microL at post-day 5 (p<0.0001) but improved to 7.9±0.5 K/microL on post-day 8. The Ptx-G-CSF group's pretreatment RBC count was 11.3±0.3 K/microL, declined to 4.8±1.1 K/microL (p<0.0001) on post-day 5 and was 7.4.±1.0 K/microL on post-day 8. The baseline hemoglobin was similar in all groups (14.6±0.4g/dL in Ptx-alone; 15.53±0.5 g/dL in Ptx+MBG; 15.2±0.9 g/dL in Ptx+G-CSF) and dropped to a nadir of about 8 g/dl in all groups on post day 5.

Platelets: The changes in platelet numbers were normalized based on each individual baseline count. Platelets increased within in all groups after Ptx; overall variation in percent change from baseline across time after Ptx treatment was significant within each group (p21 0.0001). In the Ptx-alone group, platelet levels increased to 76.5%±25.6 above baseline at post day 3 (p<0.001, Tukey's). For the Ptx+G-CSF group mean platelet levels were always significantly above baseline after post-day 3 (p<0.001, Tukey's) and in the Ptx+MBG group after day 1 (p<0.001, Tukey's).

### EXAMPLE 11

Recovery of Neutrophil and Monocyte function after Ptx treatment:

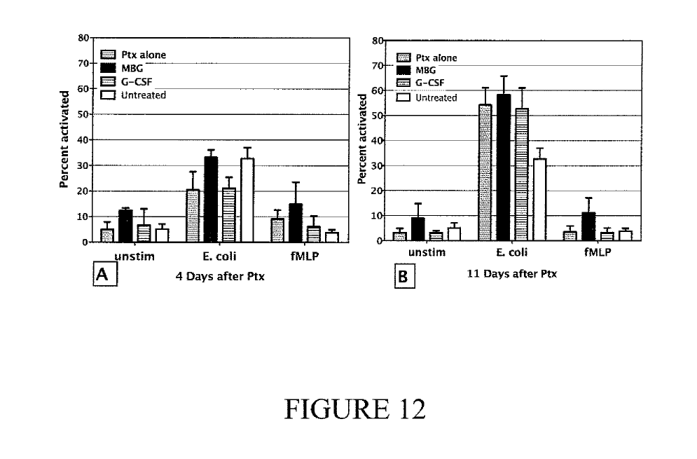

To determine the functionality of myeloid cells after Ptx treatment and the effects MBG or G-CSF treatment, production of reactive oxygen species (ROS) in Gr-1+ myeloid cells was examined. After brief exposure of whole blood to opsonized E. coli and the chemotactic peptide N-formyl-Met-Leu-Phe (fMLP) ex vivo, fluorescence of ROS positive cells was detected by the oxidation of the dihydrorhodamine substrate. Blood samples were collected from each group 4 days after the last dose of Ptx and again on post-day 11. FIG. 12, Panel A, shows the percentage of ROS+ cells in the three treatment groups on post-day 4. The responses varied significantly within each treatment group by test stimulus (p<0.0001, ANOVA, each group). The responses of the three treatment groups showed across-group variation (two-way ANOVA, p<0.001). The response to E. coli was stronger in the Ptx+MBG group compared to the Ptx-alone group or to the Ptx+G-SCF group (p<0.01 for each, Bonferroni posttest). Response to fMLP was stronger in the Ptx+MBG group compared to the Ptx+G-CSF group (p<0.05). Post-day 4 was 3 days after G-CSF injection for the Ptx+G-CSF group. The ROS responses of normal, untreated mice were also studied. As shown in FIG. 12, panel A, untreated mice produced a significantly greater ROS response to E. coli than did mice treated with Ptx alone or with Ptx+G-CSF (p<0.01 for both). In contrast ROS response in the Ptx+MBG group was equal to that of untreated mice. Interestingly the fMLP response at post-day 4 was higher in the Ptx+MBG group compared to normal untreated mice (p<0.01).

On post-day 11 the ROS response to E. coli was equivalent in all treatment groups. For each of the treatment groups significant variation across the two time points was observed (p<0.001, one-way ANOVA for each). See FIG. 12, panel B. The ROS response to E. coli increased (p<0.0001) on post-day 11 compared to 4, while response to fMLP decreased (p<0.0001) in the Ptx-alone group. Unstimulated ROS production was unchanged. For both Ptx+MBG treated and Ptx+G-CSF groups, the percentage of ROS producing cells also increased on post-day 11 compared to post-day 4 (p<0.0001) in response to E. coli. However, responses to fMLP varied overall across the groups (p <0.01 one-way ANOVA). The Ptx+MBG treated group showed a higher response to fMLP compared to both the Ptx-alone group and the Ptx+G-CSF group (p<0.05 Tukey's). See FIG. 12, panel B. The ROS response of untreated mice to E. coli was now much lower compared to each of the three treatment groups (p<0.0001).

### EXAMPLE 12

The studies described in Examples 6-11 examined for the first time the dynamics of leukocyte recovery in peripheral blood after Ptx treatment in vivo in a normal mouse. Ptx alone led to suppression of peripheral blood white blood cell counts below baseline levels for more than 8 days. Maitake mushroom beta-glucan (MBG) or G-CSF treatment stimulated earlier recovery of leukocytes and increased the numbers of neutrophils and lymphocytes above baseline by post Ptx day 5. Giving oral MBG throughout chemotherapy was as effective as a single dose of G-CSF given i.v. after the last dose of Ptx in reducing leukopenia in this model. Doses of G-CSF in the range of 10 μg/kg to 250 μg/kg have been shown to enhance peripheral blood cell counts when given to normal mice without prior chemotherapy [111, 112]. MBG enhanced hematopoietic progenitor cell CFU-GM activity two days after Ptx treatment and increased leukocyte recovery in peripheral blood two days later. It has been shown that bone marrow CFU-GM content is directly linked to recovery of peripheral blood cells [113]. In the Examples hereinabove, it has been shown that myeloid cell function is compromised by Ptx treatment and that MBG, but not G-CSF, treatment restored the oxidative burst response to normal levels after treatment. Activation of ROS activity appears to be distinct and novel property of MBG in addition to promotion of hematopoietic progenitor cell maturation and mobilization of leukocytes into the periphery.

Comparatively few investigations have used in vivo models to study hematopoiesis in both bone marrow and the peripheral blood compartments after paclitaxel chemotherapy. It has been found herein that the maximum loss of erythrocytes in peripheral blood occurred later compared to lymphoid or myeloid cells after dose-intensive Ptx. While the recovery of myeloid cells was enhanced in the Ptx treated mice that also received G-CSF or MBG, the course and magnitude of Ptx mediated suppression of RBC numbers and hemoglobin levels were not affected. Platelet numbers were strikingly increased in all mice receiving Ptx and this was also not affected by either concurrent MBG or therapeutic G-CSF.

The studies herein indicate that MBG could synergize with G-CSF in supporting functional myeloid cell recovery. MBG clearly enhanced mobilization of myeloid cells into blood since we observed a shortened time to leukocyte recovery in peripheral blood after chemotherapy. G-CSF is widely given intravenously for the collection of hematopoietic progenitor and stem cells for transplantation in neoplastic diseases. MBG could enhance the effect of G-CSF. Therefore MBG may enhance the response of poor as well as normal mobilizers to G-CSF. In summation, the Examples herein demonstrate that beta-glucans are useful in the support of bone marrow recovery after cancer chemotherapy.

### REFERENCES

71. Citron M L, Berry D A, Cirrincione C, Hudis C, Winer E P, Gradishar W J, Davidson N E, Martino S, Livingston R, Ingle J N, Perez E A, Carpenter J, Hurd D, Holland J F, Smith B L, Sartor C I, Leung E H, Abrams J, Schilsky R L, Muss H B, Norton L (2003) Randomized trial of dose-dense versus conventionally scheduled and sequential versus concurrent combination chemotherapy as postoperative adjuvant treatment of node-positive primary breast cancer: first report of Intergroup Trial C9741/Cancer and Leukemia Group B Trial 9741. J Clin Oncol 21:1431-1439
72. Fornier M, Norton L (2005) Dose-dense adjuvant chemotherapy for primary breast cancer. Breast Cancer Res 7:64-69
73. De Boer R H, Eisen T G, Ellis P A, Johnston S R, Walsh G, Ashley S, Smith I E (2002) A randomised phase II study of conventional versus accelerated infusional chemotherapy with granulocyte colony-stimulating factor support in advanced breast cancer. Ann Oncol 13:889-894
74. Osma M M, Ortuno F, Lozano M L, Gomez-Espuch J, Ayala F, Sanchez-Serrano I, Perez-Ceballos E, Moraleda J M, Vicente V (2001) Administration of post-autologous PBSCT rhG-CSF is associated with long-term low concentrations of bone marrow hematopoietic progenitor cells. Bone Marrow Transplant 27:1287-1292
75. Pape H, Orth K, Heese A, Heyll A, Kobbe G, Schmitt G, Niederbichler A D, Peiper M, Schwarz A, Boelke E (2006) G-CSF during large field radiotherapy reduces bone marrow recovery capacity. Eur J Med Res 11:322-328
76. Arnedos M, Sutherland S, Ashley S, Smith I (2008) Routine prophylactic granulocyte colony stimulating factor (GCSF) is not necessary with accelerated (dose dense) paclitaxel for early breast cancer. Breast Cancer Res Treat 112:1-4
77. Sugarman S, Wasserheit C, Hodgman E, Coglianese M, D'Alassandro A, Fournier M, Troso-Sandoval T, D'Andrea G, Drullinsky P, Lake D, George R, Mills N, Moynahan M, Smith J, Panageas K, Norton L, Hudis C (2009) A pilot study of dose-dense adjuvant paclitaxel without growth factor support for women with early breast carcinoma. Breast Cancer Res Treat 115:609-612
78. Seidman A D, Berry D, Cirrincione C, Harris L, Muss H, Marcom P K, Gipson G, Burstein H, Lake D, Shapiro C L, Ungaro P, Norton L, Winer E, Hudis C (2008) Randomized phase III trial of weekly compared with every-3-weeks paclitaxel for metastatic breast cancer, with trastuzumab for all HER-2 overexpressors and random assignment to trastuzumab or not in HER-2 nonoverexpressors: final results of Cancer and Leukemia Group B protocol 9840. J Clin Oncol 26:1642-1649
79. Untch M, Mobus V, Kuhn W, Muck B R, Thomssen C, Bauerfeind 1, Harbeck N, Werner C, Lebeau A, Schneeweiss A, Kahlert S, von Koch F, Petry K U, Wallwiener D, Kreienberg R, Albert U S, Luck H J, Hinke A, Janicke F, Konecny G E (2009) Intensive dose-dense compared with conventionally scheduled preoperative chemotherapy for high-risk primary breast cancer. J Clin Oncol 27:2938-2945
80. Horiguchi J, Rai Y, Tamura K, Taki T, Hisamatsu K, Ito Y, Seriu T, Tajima T (2009) Phase II study of weekly paclitaxel for advanced or metastatic breast cancer in Japan. Anticancer Res 29:625-630
81. Gianni L, Baselga J, Eiermann W, Porta V G, Semiglazov V, Lluch A, Zambetti M, Sabadell D, Raab G, Cussac A L, Bozhok A, Martinez-Agullo A, Greco M, Byakhov M, Lopez J J, Mansutti M, Valagussa P, Bonadonna G (2009) Phase III trial evaluating the addition of paclitaxel to doxorubicin followed by cyclophosphamide, methotrexate, and fluorouracil, as adjuvant or primary systemic therapy: European Cooperative Trial in Operable Breast Cancer. J Clin Oncol 27:2474-2481
82. Zhou J, Zhang H, Gu P, Margolick J B, Yin D, Zhang Y (2008) Cancer stem/progenitor cell active compound 8-quinolinol in combination with paclitaxel achieves an improved cure of breast cancer in the mouse model. Breast Cancer Res Treat
83. Fountzilas G, Dafni U, Dimopoulos M A, Koutras A, Skarlos D, Papakostas P, Gogas H, Bafaloukos D, Kalogera-Fountzila A, Samantas E, Briasoulis E, Pectasides D, Maniadakis N, Matsiakou F, Aravantinos G, Papadimitriou C, Karina M, Christodoulou C, Kosmidis P, Kalofonos HP (2009) A randomized phase III study comparing three anthracycline-free taxane-based regimens, as first line chemotherapy, in metastatic breast cancer: a Hellenic Cooperative Oncology Group study. Breast Cancer Res Treat 115:87-99
84. Deng G, Lin H, Seidman A, Farther M, D'Andrea G, Wesa K, Yeung S, Cunningham-Rundles S, Vickers A J, Cassileth B (2009) A phase III trial of a polysaccharide extract from Grifola frondosa (Maitake mushroom) in breast cancer patients: immunological effects. J Cancer Res Clin Oncol
85. Tsoni S V, Brown G D (2008) beta-Glucans and dectin-1. Ann N Y Acad Sci 1143:45-60
86. Brown G D, Taylor P R, Reid D M, Willment J A, Williams D L, Martinez-Pomares L, Wong S Y, Gordon S (2002) Dectin-1 is a major beta-glucan receptor on macrophages. J Exp Med 196:407-412
87. Cramer D E, Allendorf D J, Baran J T, Hansen R, Marroquin J, Li B, Ratajczak J, Ratajczak M Z, Yan J (2006) Beta-glucan enhances complement-mediated hematopoietic recovery after bone marrow injury. Blood 107:835-840
88. Ross G D, Vetvicka V, Yan J, Xia Y, Vetvickova J (1999) Therapeutic intervention with complement and beta-glucan in cancer. Immunopharmacology 42:61-74
89. Cheung N K, Modak S, Vickers A, Knuckles B (2002) Orally administered beta-glucans enhance anti-tumor effects of monoclonal antibodies. Cancer Immunol Immunother 51:557-564
90. Harada T, Kawaminami H, Miura N N, Adachi Y, Nakajima M, Yadomae T, Ohno N (2006) Mechanism of enhanced hematopoietic response by soluble beta-glucan SCG in cyclophosphamide-treated mice. Microbial Immunol 50:687-700
91. Patchen M L, MacVittie T J, Solberg B D, Souza L M (1990) Survival enhancement and hemopoietic regeneration following radiation exposure: therapeutic approach using glucan and granulocyte colony-stimulating factor. Exp Hematol 18:1042-1048
92. Hofer M, Pospisil M (1997) Glucan as stimulator of hematopoiesis in normal and gamma-irradiated mice. A survey of the authors' results. Int J Immunopharmacol 19:607-609
93. Cramer D E, Wagner S, Li B, Liu J, Hansen R, Reca R, Wu W, Surma E Z, Laber D A, Ratajczak M Z, Yan J (2008) Mobilization of hematopoietic progenitor cells by yeast-derived beta-glucan requires activation of matrix metalloproteinase-9. Stem Cells 26:1231-1240
94. Lin H, She Y H, Cassileth B R, Sirotnak F, Cunningham Rundles S (2004) Maitake beta-glucan MD-fraction enhances bone marrow colony formation and reduces doxorubicin toxicity in vitro. Int Immunopharmacol 4:91-99
95. Lin H, Cheung S W, Nesin M, Cassileth B R, Cunningham-Rundles S (2007) Enhancement of umbilical cord blood cell hematopoiesis by maitake beta-glucan is mediated by granulocyte colony-stimulating factor production. Clin Vaccine Immunol 14:21-27
96. Lin H, De Stanchina E, Zhou X K, She Y H, Hoang D, Cheung S W, Cassileth B R, Cunningham-Rundles S (2009) Maitake Beta-Glucan Enhances Umbilical Cord Blood Stem Cell Transplantation in the NOD/SCID Mouse. Exp Biol Med (Maywood)

### 97. York, W S, Darvill, A G, McNeil, M, Siwenson, T T, and Albersheim, P. Methods Enzymol., 118 (1986)3-40.

98. Merkle R K, Poppe I (1994) Carbohydrate composition analysis of glycoconjugates by gas-liquid chromatography/mass spectrometry. Methods Enzymol 230:1-15
99. Ballabh P, Simm M, Kumari J, Califano C, Aghai Z, Laborada G, Sison C, Cunningham-Rundles S (2003) Respiratory burst activity in bronchopulmonary dysplasia and changes with dexamethasone. Pediatr Pulmonol 35:392-399
100. Wani M C, Taylor H L, Wall M E, Coggon P, McPhail A T (1971) Plant antitumor agents. V I. The isolation and structure of taxol, a novel antileukemic and antitumor agent from Taxus brevifolia. J Am Chem Soc 93:2325-2327
101. Eisenhauer E A, Vermorken J B (1998) The taxoids. Comparative clinical pharmacology and therapeutic potential. Drugs 55:5-30
102. Kim-Park W K, Moore M A, Hakki Z W, Kowolik M J (1997) Activation of the neutrophil respiratory burst requires both intracellular and extracellular calcium. Ann N Y Acad Sci 832:394-404
103. Poruchynsky M S, Wang E E, Rudin C M, Blagosklonny M V, Fojo T (1998) Bcl-xL is phosphorylated in malignant cells following microtubule disruption. Cancer Res 58:3331-3338
104. Crossin K L, Carney D H (1981) Microtubule stabilization by taxol inhibits initiation of DNA synthesis by thrombin and by epidermal growth factor. Cell 27:341-350
105. Rowinsky E K, Donehower R C (1995) Paclitaxel (taxol). N Engl J Med 332:1004-1014
106. Fetterly G J, Tamburlin J M, Straubinger R M (2001) Paclitaxel pharmacodynamics: application of a mechanism-based neutropenia model. Biopharm Drug Dispos 22:251-261
107. Bulitta J B, Zhao P, Arnold R D, Kessler D R, Daifuku R, Pratt J, Luciano G, Hanauske A R, Gelderblom H, Awada A, Jusko W J (2009) Multiple-pool cell lifespan models for neutropenia to assess the population pharmacodynamics of unbound paclitaxel from two formulations in cancer patients. Cancer Chemother Pharmacol 63:1035-1048
108. Polioudaki H, Kastrinaki M C, Papadaki H A, Theodoropoulos P A (2009) Microtubule-interacting drugs induce moderate and reversible damage to human bone marrow mesenchymal stem cells. Cell Prolif 42:434-447
109. Panopoulos A D, Watowich S S (2008) Granulocyte colony-stimulating factor: molecular mechanisms of action during steady state and ‘emergency’ hematopoiesis. Cytokine 42:277-288
110. Moore M A, Warren D J (1987) Synergy of interleukin 1 and granulocyte colony-stimulating factor: in vivo stimulation of stem-cell recovery and hematopoietic regeneration following 5-fluorouracil treatment of mice. Proc Natl Acad Sci U S A 84:7134-7138
111. Molineux G, Pojda Z, Dexter T M (1990) A comparison of hematopoiesis in normal and splenectomized mice treated with granulocyte colony-stimulating factor. Blood 75:563-569
112. Lord BI, Molineux G, Pojda Z, Souza L M, Mermod J J, Dexter T M (1991) Myeloid cell kinetics in mice treated with recombinant interleukin-3, granulocyte colony-stimulating factor (CSF), or granulocyte-macrophage CSF in vivo. Blood 77:2154-2159
113. Rowley S D, Zuehlsdorf M, Braine H G, Colvin O M, Davis J, Jones R J, Saral R, Sensenbrenner L L, Yeager A, Santos G W (1987) CFU-GM content of bone marrow graft correlates with time to hematologic reconstitution following autologous bone marrow transplantation with 4-hydroperoxycyclophosphamide-purged bone marrow. Blood 70:271-275
114. Lejeune M, Sariban E, Cantinieaux B, Ferster A, Devalck C, Fondu P (1996) Defective polymorphonuclear leukocyte functions in children receiving chemotherapy for cancer are partially restored by recombinant human granulocyte colony-stimulating factor in vitro. J Infect Dis 174:800-805
115. Lejeune M, Ferster A, Cantinieaux B, Sariban E (1998) Prolonged but reversible neutrophil dysfunctions differentially sensitive to granulocyte colony-stimulating factor in children with acute lymphoblastic leukaemia. Br J Haematol 102:1284-1291
116. Hubel K, Hegener K, Schnell R, Mansmann G, Oberhauser F, Staib P, Diehl V, Engert A (1999) Suppressed neutrophil function as a risk factor for severe infection after cytotoxic chemotherapy in patients with acute nonlymphocytic leukemia. Ann Hematol 78:73-77
117. Wiles M E, Dykens J A, Wright C D (1994) Regulation of polymorphonuclear leukocyte membrane fluidity: effect of cytoskeletal modification. J Leukoc Biol 56:192-199
118. Wiles M E, Dykens J A, Wright C D (1995) Human neutrophil (PMN) oxygen radical production and the cytoskeleton. Life Sci 57:1533-1546
119. Roberts R L, Nath J, Friedman M M, Gallin J I (1982) Effects of taxol on human neutrophils. J Immunol 129:2134-2141
120. Garcia I, Pascual A, Salvador J, Conejo M C, Perea E J (1996) Effect of paclitaxel alone or in combination on the intracellular penetration and activity of quinolones in human neutrophils. J Antimicrob Chemother 38:859-863
121. Boxio R, Bossenmeyer-Pourie C, Steinckwich N, Dournon C, Nusse O (2004) Mouse bone marrow contains large numbers of functionally competent neutrophils. J Leukoc Biol 75:604-611
122. Ito K, Masuda Y, Yamasaki Y, Yokota Y, Nanba H (2009) Maitake beta-glucan enhances granulopoiesis and mobilization of granulocytes by increasing G-CSF production and modulating CXCR4/SDF-1 expression. Int Immunopharmacol
123. Hoglund M, Hakansson L, Venge P (1997) Effects of in vivo administration of G-CSF on neutrophil functions in healthy volunteers. Eur J Haematol 58:195-202
124. Romero-Benitez M M, Aguirre M V, Juaristi J A, Alvarez M A, Trifaro J M, Brandan N C (2004) In vivo erythroid recovery following paclitaxel injury: correlation between GATA-1, c-MYB, NF-E2, Epo receptor expressions, and apoptosis. Toxicol Appl Pharmacol 194:230-238
125. Juaristi J A, Aguirre M V, Carmuega R J, Romero-Benitez M, Alvarez M A, Brandan N C (2001) Hematotoxicity induced by paclitaxel: in vitro and in vivo assays during normal murine hematopoietic recovery. Methods Find Exp Clin Pharmacol 23:161-167
126. Ojeifo J O, Wu A G, Miao Y, Herscowtiz H B, Meehan K R (2000) Docetaxel-induced mobilization of hematopoietic stem cells in a murine model: kinetics, dose titration, and toxicity. Exp Hematol 28:451-459
127. Churin A A, Goldberg V E, Karpova G V, Voronova O L, Feodorova E P, Kolotova O V, Skurikhin E G, Pershina O V (2008) Reaction of bone marrow hematopoiesis to the toxic effect of paclitaxel. Bull Exp Biol Med 145:213-217
128. Pertusini E, Ratajczak J, Majka M, Vaughn D, Ratajczak M Z, Gewirtz A M (2001) Investigating the platelet-sparing mechanism of paclitaxel/carboplatin combination chemotherapy. Blood 97:638-644
129. Panaro M A, Mitolo V (1999) Cellular responses to FMLP challenging: a mini-review. Immunopharmacol Immunotoxicol 21:397-419
130. Hailer M B, Wakshull E, Filardo E J, Albina J E, Reichner J S (1999) Promotion of neutrophil chemotaxis through differential regulation of beta 1 and beta 2 integrins. J Immunol 162:6792-6799
131. Lapidot T (2001) Mechanism of human stem cell migration and repopulation of NOD/SCID and B2Mnull NOD/SOD mice. The role of SDF-1/CXCR4 interactions. Ann N Y Acad Sci 938:83-95
132. Ratajczak M Z, Wysoczynski M, Reca R, Wan W, Zuba-Surma E K, Kucia M, Ratajczak J (2008) A pivotal role of activation of complement cascade (CC) in mobilization of hematopoietic stem/progenitor cells (HSPC). Adv Exp Med Biol 632:47-60
133. Winkler I G, Levesque J P (2006) Mechanisms of hematopoietic stem cell mobilization: when innate immunity assails the cells that make blood and bone. Exp Hematol 34:996-1009
134. Reca R, Cramer D, Yan J, Laughlin M J, Janowska-Wieczorek A, Ratajczak J, Ratajczak M Z (2007) A novel role of complement in mobilization: immunodeficient mice are poor granulocyte-colony stimulating factor mobilizers because they lack complement-activating immunoglobulins. Stem Cells 25:3093-3100
135. Siegal F P, Lopez C, Hammer G S, Brown A E, Kornfeld S J, Gold J, Hassett J, Hirschman S Z, Cunningham-Rundles C, Adelsberg B R, et al. (1981) Severe acquired immunodeficiency in male homosexuals, manifested by chronic perianal ulcerative herpes simplex lesions. N Engl J Med 305:1439-1444
136. Schulof R S, Bockman R S, Garofalo J A, Cirrincione C, Cunningham-Rundles S, Fernandes G, Day N K, Pinsky C M, Incefy G S, Thaler H T, Good R A, Gupta S (1981) Multivariate analysis of T-cell functional defects and circulating serum factors in Hodgkin's disease. Cancer 48:964-973

What is claimed is:

1. A method for expanding CD34+ cells in an initial population of cells comprising hematopoietic progenitor cells and/or pluripotent hematopoietic stem cells, said method comprising culturing the initial population of cells in vitro in the presence of a beta glucan composition.

2. The method of claim 1 wherein said beta glucan composition is prepared from an organism selected from the group consisting of bacteria, yeast, mushrooms, seaweed, and grains.

3. The method of claim 1 wherein said beta glucan composition is an extract prepared from maitake.

4. The method of claim 1 wherein the initial population of cells is obtained from a sample selected from the group consisting of an umbilical cord blood sample, bone marrow, spleen, and peripheral blood.

5. The method of claim 4 wherein the initial population of cells is obtained from umbilical cord blood.

6. The method of claim 1 wherein said hematopoietic progenitor cells comprise hematopoietic committed and/or lineage-restricted progenitor cells.

7. The method of claim 1, further comprising selecting or isolating or otherwise altering the CD34+ cells after the culturing.

8. A method of promoting homing of a population of cells which comprises primitive hematopoietic progenitor cells from a donor mammal to the bone marrow of a recipient mammal, comprising culturing said population with a beta glucan composition in vitro prior to administration of the cells to said recipient mammal.

9. The method of claim 8 wherein the beta glucan composition is an extract prepared from maitake.

10. The method of claim 8 wherein said population of cells is obtained from a sample from said donor mammal selected from the group consisting of an umbilical cord blood sample, bone marrow, and a peripheral blood sample.

11. The method of claim 10 wherein said sample is an umbilical cord blood sample.

12. The method of claim 8, further comprising isolating CD34+ cells after the culturing to obtain a CD34+ enriched cell population or otherwise altering said cells for administration to the recipient mammal.

13. A method of promoting engraftment of a population of cells which comprises hematopoietic progenitor and/or stem cells from a donor mammal to the bone marrow of a recipient mammal, comprising culturing said population with a beta glucan composition in vitro prior to administration of the cells to said recipient mammal.

14. The method of claim 13 wherein the beta glucan is an extract prepared from maitake.

15. The method of claim 13 wherein said population of cells is obtained from a sample from said donor mammal selected from the group consisting of an umbilical cord blood sample, bone marrow, and a peripheral blood sample.

16. The method of claim 15 wherein said sample is an umbilical cord blood sample.

17. The method of claim 13, further comprising isolating CD34+ cells after the culturing to obtain a CD34+ enriched cell population or otherwise altering said cells for administration to the recipient mammal.

18. A method of promoting homing of a population of cells which comprises hematopoietic progenitor and/or stem cells from a donor mammal to the bone marrow of a recipient mammal, comprising administering a beta glucan composition and administering said population of cells to said recipient mammal.

19. The method of claim 18 wherein the beta glucan composition is an extract prepared from maitake.

20. The method of claim 18 wherein the beta glucan composition is administered orally.

21. The method of claim 18 wherein the beta glucan composition is administered intermixed with said population of cells.

22. The method of claim 18 wherein the beta glucan composition is administered prior to administration of said population of cells.

23. The method of claim 18 wherein the beta glucan composition is administered at the same time as the administration of the population of cells.

24. The method of claim 18 wherein the beta glucan composition is administered subsequent to the administration of the population of cells.

25. The method of claim 18 wherein the administered population of cells is obtained from a sample from said donor mammal selected from the group consisting of an umbilical cord blood sample, spleen, bone marrow, and a peripheral blood sample.

26. The method of claim 25 wherein said sample is an umbilical cord blood sample.

27. A method of promoting engraftment of a population of cells which comprises hematopoietic progenitor and/or stem cells from a donor mammal to the bone marrow of a recipient mammal, comprising administering a beta glucan composition and administering said population of cells to said recipient mammal.

28. The method of claim 27 wherein the beta glucan composition is an extract prepared from maitake.

29. The method of claim 27 wherein the beta glucan composition is administered orally.

30. The method of claim 27 wherein the beta glucan composition is administered intermixed with said population of cells.

31. The method of claim 27 wherein the beta glucan composition is administered prior to administration of said population of cells.

32. The method of claim 27 wherein the beta glucan composition is administered at the same time as the administration of the population of cells.

33. The method of claim 27 wherein the beta glucan composition is administered subsequent to the administration of the population of cells.

34. The method of claim 27 wherein the administered population of cells is obtained from a sample from said donor mammal selected from the group consisting of an umbilical cord blood sample, bone marrow, and a peripheral blood sample.

35. The method of claim 34 wherein said sample is an umbilical cord blood sample.

36. A method of reducing the hematologic toxicity of chemotherapy associated with cancer treatment in a mammal, comprising administering a beta glucan composition to said mammal in conjunction with administering a chemotherapeutic drug or drug combination.

37. The method of claim 36 wherein the beta glucan composition is an extract prepared from maitake.

38. The method of claim 36 wherein the beta glucan composition is administered orally.

39. The method of claim 36 wherein the beta glucan composition is administered before said chemotherapeutic drug or drug combination.

40. The method of claim 36 wherein the beta glucan composition is administered simultaneously with said chemotherapeutic drug or drug combination.

41. The method of claim 36 wherein the beta glucan composition is administered in the period following the administration of said chemotherapeutic drug or drug combination.

42. The method of claim 36 wherein said chemotherapeutic drug is a taxane.
